SEQUENCE LISTING (96 sequences)

SEQ ID NO: 1 - DNA, 1244 bp, Homo sapiens
  source 1..1244: mol_type = other DNA
ggatggggagataagagaagaccaggagggagttaaatagggaatgggttgggggcggcttggtaaatgtgctgggattaggctgttgcagataatgcaacaaggcttggaaggctaacctgggactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgcgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 2 - DNA, 1244 bp, Homo sapiens
  source 1..1244: mol_type = other DNA
ggatggggagataagagaagaccaggagggagttaaatagggaatgggttgggggcggcttggtaaatgtgctgggattaggctgttgcagataatgcaacaaggcttggaaggctaacctgggactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 3 - DNA, 1179 bp, Macaca fascicularis
  source 1..1179: mol_type = other DNA
atgagctcaggcgcctctagaaagtgtagctgggacctcgggaagccctggcctccagactggccaatcacaggcaggaagatgaaggttctgtgggctgcgttgctggtcacattcctggcaggatgccaggccaaggtggagcaaccggtggagccagagacggaacccgagcttcgccagcaggctgaggggcagagcggccagccctgggagctggcactgggtcgcttttgggattacctgcgctgggtgcagacactgtctgagcaggtgcaggaggagctgctcagcccccaggtcacccaggaactgacgacgctgatggatgagaccatgaaggagttgaaggcctacaaatcggaactggaggaacagctgagcccggtggcggaggagacgcgggcacggctgtccaaggagctgcaggcggcgcaggcccggctgggtgccgacatggaggacgtgcgcagccgcctggtgcagtaccgcagcgaggtgcaggccatgctgggccagagtaccgaggagctgcgggcgcgcctcgcctcccacctgcgcaagctgcgcaagcggctcctccgcgatgctgatgacctgcagaagcgcctggcagtgtatcaggccggggcccgcgagggcgccgagcgcggggtcagcgccatccgcgagcgcctgggacccctggtggagcagggccgcgtgcgggccgccactgtgggctccctggccagccagccgcttcaggagcgggcccaggccttgggtgagcggcttcgcgcacggatggaggagatgggcagccggacccgcgaccgcctggacgaggtgaaggagcaggtggcggaggtgcgcgccaagctggaggaacaggcccagcagataagcctgcaggccgaggccttccaggcccgcctcaagagctggttcgagcccctcgtggaagatatgcagcgccagtgggctgggctggtggagaaggtgcaggctgccgtgggcgccagcaccgcccctgtgcccagcgacaatcactgaacgcccaggcctacagccatgcgacccgactccaccccatgcctcctctctccgctcagcctgcagcgggagaccctgtccccgccccagccgtcctccaggggtgggccctagtttaataaagattcgccaagtttcaccgca

SEQ ID NO: 4 - DNA, 20 bp, synthetic construct
  misc_feature 1..20: note = Synthetic oligonucleotide
  source 1..20: mol_type = other DNA
gcacgtcctccatgtccgcg

SEQ ID NO: 5 - DNA, 20 bp, synthetic construct
  misc_feature 1..20: note = Synthetic oligonucleotide
  source 1..20: mol_type = other DNA
cgcacgtcctccatgtccgc

SEQ ID NO: 6 - DNA, 19 bp, synthetic construct
  misc_feature 1..19: note = Synthetic oligonucleotide
  source 1..19: mol_type = other DNA
gcacgtcctccatgtccgc

SEQ ID NO: 7 - DNA, 20 bp, synthetic construct
  misc_feature 1..20: note = Synthetic oligonucleotide
  source 1..20: mol_type = other DNA
cgcgcacgtcctccatgtcc

SEQ ID NO: 8 - DNA, 20 bp, synthetic construct
  misc_feature 1..20: note = Synthetic oligonucleotide
  source 1..20: mol_type = other DNA
gcgcacgtcctccatgtccg

SEQ ID NO: 9 - DNA, 19 bp, synthetic construct
  misc_feature 1..19: note = Synthetic oligonucleotide
  source 1..19: mol_type = other DNA
cgcacgtcctccatgtccg

SEQ ID NO: 10 - DNA, 18 bp, synthetic construct
  misc_feature 1..18: note = Synthetic oligonucleotide
  source 1..18: mol_type = other DNA
gcacgtcctccatgtccg

SEQ ID NO: 11 - DNA, 19 bp, synthetic construct
  misc_feature 1..19: note = Synthetic oligonucleotide
  source 1..19: mol_type = other DNA
cgcgcacgtcctccatgtc

SEQ ID NO: 12 - DNA, 19 bp, synthetic construct
  misc_feature 1..19: note = Synthetic oligonucleotide
  source 1..19: mol_type = other DNA
gcgcacgtcctccatgtcc

SEQ ID NO: 13 - DNA, 18 bp, synthetic construct
  misc_feature 1..18: note = Synthetic oligonucleotide
  source 1..18: mol_type = other DNA
cgcacgtcctccatgtcc

SEQ ID NO: 14 - DNA, 17 bp, synthetic construct
  misc_feature 1..17: note = Synthetic oligonucleotide
  source 1..17: mol_type = other DNA
gcacgtcctccatgtcc

SEQ ID NO: 15 - DNA, 20 bp, synthetic construct
  misc_feature 1..20: note = Synthetic oligonucleotide
  source 1..20: mol_type = other DNA
ccgcgcacgtcctccatgtc

SEQ ID NO: 16 - DNA, 18 bp, synthetic construct
  misc_feature 1..18: note = Synthetic oligonucleotide
  source 1..18: mol_type = other DNA
cgcgcacgtcctccatgt

SEQ ID NO: 17 - DNA, 18 bp, synthetic construct
  misc_feature 1..18: note = Synthetic oligonucleotide
  source 1..18: mol_type = other DNA
gcgcacgtcctccatgtc

SEQ ID NO: 18 - DNA, 17 bp, synthetic construct
  misc_feature 1..17: note = Synthetic oligonucleotide
  source 1..17: mol_type = other DNA
cgcacgtcctccatgtc

SEQ ID NO: 19 - DNA, 16 bp, synthetic construct
  misc_feature 1..16: note = Synthetic oligonucleotide
  source 1..16: mol_type = other DNA
gcacgtcctccatgtc

SEQ ID NO: 20 - DNA, 20 bp, synthetic construct
  misc_feature 1..20: note = Synthetic oligonucleotide
  source 1..20: mol_type = other DNA
gccgcgcacgtcctccatgt

SEQ ID NO: 21 - DNA, 19 bp, synthetic construct
  misc_feature 1..19: note = Synthetic oligonucleotide
  source 1..19: mol_type = other DNA
ccgcgcacgtcctccatgt

SEQ ID NO: 22 - DNA, 17 bp, synthetic construct
  misc_feature 1..17: note = Synthetic oligonucleotide
  source 1..17: mol_type = other DNA
cgcgcacgtcctccatg

SEQ ID NO: 23 - DNA, 17 bp, synthetic construct
  misc_feature 1..17: note = Synthetic oligonucleotide
  source 1..17: mol_type = other DNA
gcgcacgtcctccatgt

SEQ ID NO: 24 - DNA, 16 bp, synthetic construct
  misc_feature 1..16: note = Synthetic oligonucleotide
  source 1..16: mol_type = other DNA
cgcacgtcctccatgt

SEQ ID NO: 25 - DNA, 14 bp, synthetic construct
  misc_feature 1..14: note = Synthetic oligonucleotide
  source 1..14: mol_type = other DNA
gcacgtcctccatg

SEQ ID NO: 26 - DNA, 20 bp, synthetic construct
  misc_feature 1..20: note = Synthetic oligonucleotide
  source 1..20: mol_type = other DNA
ggccgcgcacgtcctccatg

SEQ ID NO: 27 - DNA, 19 bp, synthetic construct
  misc_feature 1..19: note = Synthetic oligonucleotide
  source 1..19: mol_type = other DNA
gccgcgcacgtcctccatg

SEQ ID NO: 28 - DNA, 18 bp, synthetic construct
  misc_feature 1..18: note = Synthetic oligonucleotide
  source 1..18: mol_type = other DNA
ccgcgcacgtcctccatg

SEQ ID NO: 29 - DNA, 16 bp, synthetic construct
  misc_feature 1..16: note = Synthetic oligonucleotide
  source 1..16: mol_type = other DNA
gcgcacgtcctccatg

SEQ ID NO: 30 - DNA, 20 bp, synthetic construct
  misc_feature 1..20: note = Synthetic oligonucleotide
  source 1..20: mol_type = other DNA
cggccgcgcacgtcctccat

SEQ ID NO: 31 - DNA, 19 bp, synthetic construct
  misc_feature 1..19: note = Synthetic oligonucleotide
  source 1..19: mol_type = other DNA
ggccgcgcacgtcctccat

SEQ ID NO: 32 - DNA, 18 bp, synthetic construct
  misc_feature 1..18: note = Synthetic oligonucleotide
  source 1..18: mol_type = other DNA
gccgcgcacgtcctccat

SEQ ID NO: 33 - DNA, 17 bp, synthetic construct
  misc_feature 1..17: note = Synthetic oligonucleotide
  source 1..17: mol_type = other DNA
ccgcgcacgtcctccat

SEQ ID NO: 34 - DNA, 16 bp, synthetic construct
  misc_feature 1..16: note = Synthetic oligonucleotide
  source 1..16: mol_type = other DNA
cgcgcacgtcctccat

SEQ ID NO: 35 - DNA, 20 bp, synthetic construct
  misc_feature 1..20: note = Synthetic oligonucleotide
  source 1..20: mol_type = other DNA
gcggccgcgcacgtcctcca

SEQ ID NO: 36 - DNA, 17 bp, synthetic construct
  misc_feature 1..17: note = Synthetic oligonucleotide
  source 1..17: mol_type = other DNA
cggccgcgcacgtcctc

SEQ ID NO: 37 - DNA, 18 bp, synthetic construct
  misc_feature 1..18: note = Synthetic oligonucleotide
  source 1..18: mol_type = other DNA
ggccgcgcacgtcctcca

SEQ ID NO: 38 - DNA, 17 bp, synthetic construct
  misc_feature 1..17: note = Synthetic oligonucleotide
  source 1..17: mol_type = other DNA
gccgcgcacgtcctcca

SEQ ID NO: 39 - DNA, 16 bp, synthetic construct
  misc_feature 1..16: note = Synthetic oligonucleotide
  source 1..16: mol_type = other DNA
ccgcgcacgtcctcca

SEQ ID NO: 40 - DNA, 13 bp, synthetic construct
  misc_feature 1..13: note = Synthetic oligonucleotide
  source 1..13: mol_type = other DNA
gcgcacgtcctcc

SEQ ID NO: 41 - DNA, 20 bp, synthetic construct
  misc_feature 1..20: note = Synthetic oligonucleotide
  source 1..20: mol_type = other DNA
ggcggccgcgcacgtcctcc

SEQ ID NO: 42 - DNA, 19 bp, synthetic construct
  misc_feature 1..19: note = Synthetic oligonucleotide
  source 1..19: mol_type = other DNA
gcggccgcgcacgtcctcc

SEQ ID NO: 43 - DNA, 18 bp, synthetic construct
  misc_feature 1..18: note = Synthetic oligonucleotide
  source 1..18: mol_type = other DNA
cggccgcgcacgtcctcc

SEQ ID NO: 44 - DNA, 17 bp, synthetic construct
  misc_feature 1..17: note = Synthetic oligonucleotide
  source 1..17: mol_type = other DNA
ggccgcgcacgtcctcc

SEQ ID NO: 45 - DNA, 16 bp, synthetic construct
  misc_feature 1..16: note = Synthetic oligonucleotide
  source 1..16: mol_type = other DNA
gccgcgcacgtcctcc

SEQ ID NO: 46 - DNA, 13 bp, synthetic construct
  misc_feature 1..13: note = Synthetic oligonucleotide
  source 1..13: mol_type = other DNA
cgcgcacgtcctc

SEQ ID NO: 47 - DNA, 20 bp, synthetic construct
  misc_feature 1..20: note = Synthetic oligonucleotide
  source 1..20: mol_type = other DNA
aggcggccgcgcacgtcctc

SEQ ID NO: 48 - DNA, 19 bp, synthetic construct
  misc_feature 1..19: note = Synthetic oligonucleotide
  source 1..19: mol_type = other DNA
ggcggccgcgcacgtcctc

SEQ ID NO: 49 - DNA, 18 bp, synthetic construct
  misc_feature 1..18: note = Synthetic oligonucleotide
  source 1..18: mol_type = other DNA
gcggccgcgcacgtcctc

SEQ ID NO: 50 - DNA, 16 bp, synthetic construct
  misc_feature 1..16: note = Synthetic oligonucleotide
  source 1..16: mol_type = other DNA
ggccgcgcacgtcctc

SEQ ID NO: 51 - DNA, 20 bp, synthetic construct
  misc_feature 1..20: note = Synthetic oligonucleotide
  source 1..20: mol_type = other DNA
caggcggccgcgcacgtcct

SEQ ID NO: 52 - DNA, 19 bp, synthetic construct
  misc_feature 1..19: note = Synthetic oligonucleotide
  source 1..19: mol_type = other DNA
aggcggccgcgcacgtcct

SEQ ID NO: 53 - DNA, 18 bp, synthetic construct
  misc_feature 1..18: note = Synthetic oligonucleotide
  source 1..18: mol_type = other DNA
ggcggccgcgcacgtcct

SEQ ID NO: 54 - DNA, 17 bp, synthetic construct
  misc_feature 1..17: note = Synthetic oligonucleotide
  source 1..17: mol_type = other DNA
gcggccgcgcacgtcct

SEQ ID NO: 55 - DNA, 16 bp, synthetic construct
  misc_feature 1..16: note = Synthetic oligonucleotide
  source 1..16: mol_type = other DNA
cggccgcgcacgtcct

SEQ ID NO: 56 - DNA, 20 bp, synthetic construct
  misc_feature 1..20: note = Synthetic oligonucleotide
  source 1..20: mol_type = other DNA
ccaggcggccgcgcacgtcc

SEQ ID NO: 57 - DNA, 19 bp, synthetic construct
  misc_feature 1..19: note = Synthetic oligonucleotide
  source 1..19: mol_type = other DNA
caggcggccgcgcacgtcc

SEQ ID NO: 58 - DNA, 18 bp, synthetic construct
  misc_feature 1..18: note = Synthetic oligonucleotide
  source 1..18: mol_type = other DNA
aggcggccgcgcacgtcc

SEQ ID NO: 59 - DNA, 17 bp, synthetic construct
  misc_feature 1..17: note = Synthetic oligonucleotide
  source 1..17: mol_type = other DNA
ggcggccgcgcacgtcc

SEQ ID NO: 60 - DNA, 16 bp, synthetic construct
  misc_feature 1..16: note = Synthetic oligonucleotide
  source 1..16: mol_type = other DNA
gcggccgcgcacgtcc

SEQ ID NO: 61 - DNA, 20 bp, synthetic construct
  misc_feature 1..20: note = Synthetic oligonucleotide
  source 1..20: mol_type = other DNA
accaggcggccgcgcacgtc

SEQ ID NO: 62 - DNA, 19 bp, synthetic construct
  misc_feature 1..19: note = Synthetic oligonucleotide
  source 1..19: mol_type = other DNA
ccaggcggccgcgcacgtc

SEQ ID NO: 63 - DNA, 18 bp, synthetic construct
  misc_feature 1..18: note = Synthetic oligonucleotide
  source 1..18: mol_type = other DNA
caggcggccgcgcacgtc

SEQ ID NO: 64 - DNA, 17 bp, synthetic construct
  misc_feature 1..17: note = Synthetic oligonucleotide
  source 1..17: mol_type = other DNA
aggcggccgcgcacgtc

SEQ ID NO: 65 - DNA, 16 bp, synthetic construct
  misc_feature 1..16: note = Synthetic oligonucleotide
  source 1..16: mol_type = other DNA
ggcggccgcgcacgtc

SEQ ID NO: 66 - DNA, 17 bp, synthetic construct
  misc_feature 1..17: note = Synthetic oligonucleotide
  source 1..17: mol_type = other DNA
caggcggccgcgcacgt

SEQ ID NO: 67 - DNA, 20 bp, synthetic construct
  misc_feature 1..20: note = Synthetic oligonucleotide
  source 1..20: mol_type = other DNA
caccaggcggccgcgcacgt

SEQ ID NO: 68 - DNA, 19 bp, synthetic construct
  misc_feature 1..19: note = Synthetic oligonucleotide
  source 1..19: mol_type = other DNA
accaggcggccgcgcacgt

SEQ ID NO: 69 - DNA, 18 bp, synthetic construct
  misc_feature 1..18: note = Synthetic oligonucleotide
  source 1..18: mol_type = other DNA
ccaggcggccgcgcacgt

SEQ ID NO: 70 - DNA, 16 bp, synthetic construct
  misc_feature 1..16: note = Synthetic oligonucleotide
  source 1..16: mol_type = other DNA
aggcggccgcgcacgt

SEQ ID NO: 71 - DNA, 20 bp, synthetic construct
  misc_feature 1..20: note = Synthetic oligonucleotide
  source 1..20: mol_type = other DNA
gcaccaggcggccgcgcacg

SEQ ID NO: 72 - DNA, 19 bp, synthetic construct
  misc_feature 1..19: note = Synthetic oligonucleotide
  source 1..19: mol_type = other DNA
caccaggcggccgcgcacg

SEQ ID NO: 73 - DNA, 18 bp, synthetic construct
  misc_feature 1..18: note = Synthetic oligonucleotide
  source 1..18: mol_type = other DNA
accaggcggccgcgcacg

SEQ ID NO: 74 - DNA, 17 bp, synthetic construct
  misc_feature 1..17: note = Synthetic oligonucleotide
  source 1..17: mol_type = other DNA
ccaggcggccgcgcacg

SEQ ID NO: 75 - DNA, 16 bp, synthetic construct
  misc_feature 1..16: note = Synthetic oligonucleotide
  source 1..16: mol_type = other DNA
caggcggccgcgcacg

SEQ ID NO: 76 - DNA, 20 bp, synthetic construct
  misc_feature 1..20: note = Synthetic oligonucleotide
  source 1..20: mol_type = other DNA
tgcaccaggcggccgcgcac

SEQ ID NO: 77 - DNA, 19 bp, synthetic construct
  misc_feature 1..19: note = Synthetic oligonucleotide
  source 1..19: mol_type = other DNA
gcaccaggcggccgcgcac

SEQ ID NO: 78 - DNA, 18 bp, synthetic construct
  misc_feature 1..18: note = Synthetic oligonucleotide
  source 1..18: mol_type = other DNA
caccaggcggccgcgcac

SEQ ID NO: 79 - DNA, 17 bp, synthetic construct
  misc_feature 1..17: note = Synthetic oligonucleotide
  source 1..17: mol_type = other DNA
accaggcggccgcgcac

SEQ ID NO: 80 - DNA, 16 bp, synthetic construct
  misc_feature 1..16: note = Synthetic oligonucleotide
  source 1..16: mol_type = other DNA
ccaggcggccgcgcac

SEQ ID NO: 81 - DNA, 20 bp, synthetic construct
  misc_feature 1..20: note = Synthetic oligonucleotide
  source 1..20: mol_type = other DNA
ctgcaccaggcggccgcgca

SEQ ID NO: 82 - DNA, 19 bp, synthetic construct
  misc_feature 1..19: note = Synthetic oligonucleotide
  source 1..19: mol_type = other DNA
tgcaccaggcggccgcgca

SEQ ID NO: 83 - DNA, 18 bp, synthetic construct
  misc_feature 1..18: note = Synthetic oligonucleotide
  source 1..18: mol_type = other DNA
gcaccaggcggccgcgca

SEQ ID NO: 84 - DNA, 17 bp, synthetic construct
  misc_feature 1..17: note = Synthetic oligonucleotide
  source 1..17: mol_type = other DNA
caccaggcggccgcgca

SEQ ID NO: 85 - DNA, 16 bp, synthetic construct
  misc_feature 1..16: note = Synthetic oligonucleotide
  source 1..16: mol_type = other DNA
accaggcggccgcgca

SEQ ID NO: 86 - DNA, 20 bp, synthetic construct
  misc_feature 1..20: note = Synthetic oligonucleotide
  source 1..20: mol_type = other DNA
actgcaccaggcggccgcgc

SEQ ID NO: 87 - DNA, 19 bp, synthetic construct
  misc_feature 1..19: note = Synthetic oligonucleotide
  source 1..19: mol_type = other DNA
ctgcaccaggcggccgcgc

SEQ ID NO: 88 - DNA, 18 bp, synthetic construct
  misc_feature 1..18: note = Synthetic oligonucleotide
  source 1..18: mol_type = other DNA
tgcaccaggcggccgcgc

SEQ ID NO: 89 - DNA, 17 bp, synthetic construct
  misc_feature 1..17: note = Synthetic oligonucleotide
  source 1..17: mol_type = other DNA
gcaccaggcggccgcgc

SEQ ID NO: 90 - DNA, 16 bp, synthetic construct
  misc_feature 1..16: note = Synthetic oligonucleotide
  source 1..16: mol_type = other DNA
caccaggcggccgcgc

SEQ ID NO: 91 - DNA, 14 bp, synthetic construct
  misc_feature 1..14: note = Synthetic oligonucleotide
  source 1..14: mol_type = other DNA
accaggcggccgcg

SEQ ID NO: 92 - DNA, 20 bp, synthetic construct
  misc_feature 1..20: note = Synthetic oligonucleotide
  source 1..20: mol_type = other DNA
tactgcaccaggcggccgcg

SEQ ID NO: 93 - DNA, 19 bp, synthetic construct
  misc_feature 1..19: note = Synthetic oligonucleotide
  source 1..19: mol_type = other DNA
actgcaccaggcggccgcg

SEQ ID NO: 94 - DNA, 18 bp, synthetic construct
  misc_feature 1..18: note = Synthetic oligonucleotide
  source 1..18: mol_type = other DNA
ctgcaccaggcggccgcg

SEQ ID NO: 95 - DNA, 17 bp, synthetic construct
  misc_feature 1..17: note = Synthetic oligonucleotide
  source 1..17: mol_type = other DNA
tgcaccaggcggccgcg

SEQ ID NO: 96 - DNA, 16 bp, synthetic construct
  misc_feature 1..16: note = Synthetic oligonucleotide
  source 1..16: mol_type = other DNA
gcaccaggcggccgcg

(19) United States
(12) Patent Application Publication
(10) Pub. No.: US 2024/0294915 A1
(43) Pub. Date: Sep. 5, 2024

(54) Oligonucleotides for Modulating Apolipoprotein E4 Expression

(71) Applicant: HOFFMANN-LA ROCHE INC., Little Falls, NJ (US)

(72) Inventors: Helle NYMARK, Farum (DK); Lykke PEDERSEN, København NV (DK); Lynette FOO, Riehen (CH); Anja MOELHART HOEG, Hillerød (DK); Lukasz KIELPINSKI, Riehen (CH)

(21) Appl. No.: 18/652,148

(22) Filed: May 1, 2024

Related U.S. Application Data
(63) Continuation of application No. PCT/EP2022/080478, filed on Nov. 2, 2022.

(30) Foreign Application Priority Data
Nov. 3, 2021 (EP) 21206278.0

(51) Int. Cl. C12N 15/113

(52) U.S. Cl. CPC C12N 15/113; C12N 2310/11; C12N 2310/315; C12N 2310/3231; C12N 2310/3341; C12N 2310/351

(57) ABSTRACT

The present invention relates to antisense oligonucleotides that are capable of modulating expression of Apolipoprotein E4 (ApoE4) in a target cell. The oligonucleotides can hybridize to APOE ε4 mRNA. The present invention further relates to conjugates of the oligonucleotide and pharmaceutical compositions and methods for treatment of, for example, Alzheimer's disease (AD), fronto-temporal dementia (FTD), Pick's disease (PiD), progressive supranuclear palsy (PSP), movement disorders such as Parkinson's disease (PD), dementia with Lewy Bodies, dementia in Down's Syndrome, and Niemann-Pick Type C1 Disease.

### FIELD OF INVENTION

The present invention relates to oligonucleotides (oligomers) that are complementary to APOE ε4 nucleic acids such as mRNA transcripts and useful for reducing the expression of Apolipoprotein E4 (ApoE4). Reduction of APOE ε4 transcripts and/or ApoE4 protein expression is beneficial for a range of medical disorders including, but not limited to, Alzheimer's disease (AD), fronto-temporal dementia (FTD), Pick's disease (PiD), progressive supranuclear palsy (PSP), movement disorders such as Parkinson's disease (PD), dementia with Lewy Bodies, dementia in Down's Syndrome, and Niemann-Pick Type C1 Disease.

### BACKGROUND

Apolipoprotein E (ApoE) is a lipoprotein involved in the binding of lipids such as cholesterol and phospholipids for the purpose of lipid transport. ApoE is largely produced by the hepatocytes and Kupffer cells of the liver as well as small amounts from the adrenal glands and adipose tissue. In the central nervous system, the main producers of ApoE are the astrocytes and microglia in the healthy nervous system and in the diseased state microglia and neurons contribute more to ApoE production.

ApoE is encoded by the APOE gene (OMIM 107741), which is located on chromosome 19 and which harbours two common single-nucleotide polymorphisms (SNPs); rs429358 and rs7412. These result in the three main isoforms of ApoE; ApoE2, ApoE3 and ApoE4, which differ in the amino acids at positions 112 and 158 in the protein. In ApoE3, they are occupied by a cysteine and arginine, respectively, whereas in ApoE2 and ApoE4, both sites are occupied by cysteine and arginine, respectively.

The amino acid differences affect the conformation of the ApoE isoforms and influence their ability to bind different lipids and proteins. ApoE4 has, for instance, a higher propensity to bind to heparin sulphate binding proteins and very low-density lipoproteins and reduced interactions with the LDL receptor LRP1, resulting in reduced clearance of amyloid plaques. Preclinical studies have also shown that ApoE4 may accelerate blood-brain barrier (BBB) breakdown, loss of cerebral blood flow, neuronal loss and behavioral deficits independently of amyloid-β (Montagne et al., Nat. Aging 2021;1;506-20). It has further been demonstrated that the presence of ApoE4 in the P301S mouse model (a model for tauopathies) worsens the already extensive tau-mediated neurodegeneration in a manner that was dependent upon astrocytic and microglial reactivity (Shi et al., Nature 2017;549(7673):523-527). Further studies showed that selective removal of ApoE4 in astrocytes in the same mouse model alleviated tau-mediated neurodegeneration.

The most predominant isoform of ApoE within the general population is ApoE3, whereas three percent (3%) of the world's population are homozygous for ApoE4 and 14% carry at least one copy of ApoE4. The proportion of AD patients harbouring at least one copy of ApoE4, however, is higher than in the general population at 37%. ApoE4 has also been associated with higher amyloid positivity in both normal and in mild cognitively impaired patients and with an increased risk of developing late-onset AD.

ApoE4 has been implicated in other diseases and disorders than AD. The presence of ApoE4 also lowers the age at which FTD associated neurodegeneration occurs. In PD, ApoE4 homozygous PD patients have demonstrated a faster rate of cognitive decline compared to other ApoE genotypes and PD patients have been found to develop dementia earlier in an ApoE4 copy number-dependent manner. In PiD, a dementing disorder with tau protein associated neuropathology, the ApoE4 allele has been found to be overrepresented. ApoE4 is also reported to lower the age at which dementia occurs in Down's Syndrome. In Nieman's Pick-Type C Disease, the severity of the disease is exacerbated if a patient harbours the ApoE4 allele. The ApoE4 allele frequency has also been shown to be higher in patients suffering from PSP with AD compared to patients with only PSP.

Robust and effective agents for the treatment of these and other diseases and disorders associated with ApoE4 are greatly needed.

### OBJECTIVE OF THE INVENTION

It is an objective of the present invention to provide antisense oligonucleotides, including gapmer oligonucleotides, which target APOE ε4 nucleic acids such as mRNA and reduce ApoE4 expression in target cells in vivo and in vitro.

It is also an objective to provide such antisense oligonucleotides which are more capable of reducing ApoE4 expression than ApoE3 expression.

It is also an objective to provide such antisense oligonucleotides for use in methods of treating or preventing diseases and disorders associated with ApoE4, including AD.

### SUMMARY OF THE INVENTION

The present invention relates to oligonucleotides targeting an ApoE4-encoding nucleic acid and which are thereby capable of modulating the expression of ApoE4 in a target cell. Oligonucleotides targeting the segment corresponding to positions 516 to 556 of SEQ ID NO:1 are identified, specifically oligonucleotides whose target sequence includes the residue corresponding to residue 535 of SEQ ID NO:1, the site of the polymorphism which distinguishes APOE ε4 from APOE ε3.

Accordingly, the invention provides for oligonucleotides of 8 to 50, such as 10 to 30, nucleotides in length which comprise a contiguous nucleotide sequence of at least 10 nucleotides in length with at least 80% complementarity to a target sequence of an APOE ε4 nucleic acid which includes the residue corresponding to residue 535 in SEQ ID NO: 1.

The oligonucleotide can be an antisense oligonucleotide, preferably with a gapmer design. Preferably, the oligonucleotide is capable of reducing the expression of ApoE4 by cleavage of a target nucleic acid. The cleavage is preferably achieved via nuclease recruitment. Preferably, the oligonucleotide is capable of reducing the expression of ApoE4 more than it reduces the expression of ApoE3.

In a further aspect, the invention provides pharmaceutical compositions comprising the oligonucleotides of the invention and pharmaceutically acceptable diluents, carriers, salts and/or adjuvants.

In a further aspect, the invention provides methods for modulation of ApoE4 expression in a target cell which is expressing ApoE4, by administering an oligonucleotide or composition of the invention in an effective amount to said cell. The methods include in vivo and in vitro methods.

In a further aspect the invention provides methods for treating or preventing a disease, disorder or dysfunction associated with in vivo activity or expression level of ApoE4 comprising administering a therapeutically or prophylactically effective amount of the oligonucleotide of the invention to a subject suffering from or at risk for the disease, disorder or dysfunction.

In a particular aspect the oligonucleotide or composition of the invention is used for the treatment or prevention of AD.

### DEFINITIONS

### Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides, such as 2′ sugar modified nucleosides.

### Antisense Oligonucleotides

The term “antisense oligonucleotide” as used herein is defined as oligonucleotides capable of modulating expression of a target gene by hybridizing to a target nucleic acid, in particular to a contiguous sequence on a target nucleic acid. The antisense oligonucleotides may be provided in single-stranded form, double-stranded form, essentially single-stranded form, or essentially double-stranded form. For example, antisense oligonucleotides provided as not essentially double-stranded, and which are therefore not siRNAs or shRNAs, are contemplated. Preferably, such antisense oligonucleotides are single stranded. Antisense oligonucleotides provided in essentially double-stranded form, such as in duplex form, are also contemplated. Single stranded oligonucleotides can also form hairpins or intermolecular duplex structures (duplex between two molecules of the same oligonucleotide), as long as the degree of intra or inter self-complementarity is more than about 50% across of the full length of the oligonucleotide.

### Contiguous Nucleotide Sequence

The term “contiguous nucleotide sequence” refers to the region of the oligonucleotide which is complementary to the target nucleic acid or target sequence. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence, such as a F-G-F′ gapmer region, and may optionally comprise further nucleotide(s), for example a nucleotide linker region which may be used to attach a functional group to the contiguous nucleotide sequence. The nucleotide linker region may or may not be complementary to the target nucleic acid. It is understood that the contiguous nucleotide sequence of the oligonucleotide cannot be longer than the oligonucleotide as such and that the oligonucleotide cannot be shorter than the contiguous nucleotide sequence.

### Nucleotides

Nucleotides are the building blocks of oligonucleotides and polynucleotides, and for the purposes of the present invention include both naturally occurring and non-naturally occurring nucleotides. In nature, nucleotides such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprises a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

### Modified Internucleoside Linkage

The term “modified internucleoside linkage” is defined as generally understood by the skilled person as linkages other than phosphodiester (PO) linkages, that covalently couples two nucleosides together. The oligonucleotides of the invention may therefore comprise modified internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use and may serve to protect against nuclease cleavage at regions of DNA or RNA nucleosides in the oligonucleotide of the invention, for example within the gap region G of a gapmer oligonucleotide, as well as in regions of modified nucleosides, such as region F and F′.

In an embodiment, the oligonucleotide comprises one or more internucleoside linkages modified from the natural phosphodiester, such as one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 75%, such as at least 80% or such as at least 90% of the internucleoside linkages in the oligonucleotide, or contiguous nucleotide sequence thereof, are nuclease resistant internucleoside linkages. In some embodiments all of the internucleoside linkages of the oligonucleotide, or contiguous nucleotide sequence thereof, are nuclease resistant internucleoside linkages. It will be recognized that, in some embodiments the nucleosides which link the oligonucleotide of the invention to a non-nucleotide functional group, such as a conjugate, may be phosphodiester.

Modified internucleoside linkages may be selected from the group comprising phosphorothioate, diphosphorothioate and boranophosphate. In some embodiments, the modified internucleoside linkages are compatible with the RNaseH recruitment of the oligonucleotide of the invention, for example phosphorothioate, diphosphorothioate or boranophosphate.

In some embodiments, the oligonucleotide comprises one or more neutral internucleoside linkage, particularly an internucleoside linkage selected from phosphotriester, methylphosphonate, MMI, amide-3, formacetal or thioformacetal.

Further internucleoside linkages are disclosed in WO2009/124238 (incorporated herein by reference). In an embodiment the internucleoside linkage is selected from linkers disclosed in WO2007/031091 (incorporated herein by reference). Particularly, the internucleoside linkage may be selected from —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, 0-PO(OCH₃)-0-, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, —NR^{H}—CO—NR^{H}—, and/or the internucleoside linker may be selected form the group consisting of: —O—CO—O—, —O—CO—NR^{H}, —NR^{H}—CO—CH₂—, —O—CH₂—CO—NR^{H}, —O—CH₂—CH₂—NR^{H}, —CO—NR^{H}—CH₂—, —CH₂—NR^{H}CO—, —O—CH₂—CH₂—S—, —S—CH₂—CH₂—O—, —S—CH₂—CH₂—S—, —CH₂—SO₂—CH₂—, —CH₂—CO—NR^{H}—, —O—CH₂—CH₂—NR^{H}—CO—, —CH₂—NCH₃—O—CH₂—, where R^{H} is selected from hydrogen and C1-4-alkyl.

### Phosphorothioate Internucleoside Linkages

One preferred modified internucleoside linkage is phosphorothioate. Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 80% or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

Nuclease resistant linkages, such as phosphorothioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F′, which the internucleoside linkage in region G may be fully phosphorothioate.

Advantageously, all the internucleoside linkages in the contiguous nucleotide sequence of the oligonucleotide are phosphorothioate linkages.

It is recognized that, as disclosed in EP2 742 135, antisense oligonucleotide may comprise other internucleoside linkages (other than phosphodiester and phosphorothioate), for example alkyl phosphonate/methyl phosphonate internucleosides, which according to EP2742135 may for example be tolerated in an otherwise DNA phosphorothioate gap region.

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases but are functional during nucleic acid hybridization. In this context “nucleobase” refers to both naturally occurring nucleobases such as adenine, guanine, cytosine, thymine, uracil, xanthine and hypoxanthine, as well as non-naturally occurring variants. Such variants are for example described in Hirao et al (2012) Accounts of Chemical Research vol 45, page 2055, in Bergstrom (2009) Current Protocols in Nucleic Acid Chemistry Suppl. 37 1.4.1, or in PCT/EP2021/065266 incorporated herein by reference.

In some embodiments the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′-thio-thymine, inosine, diaminopurine, 6-aminopurine, 2-aminopurine, 2,6-diaminopurine, 7-deaza-8-aza guanine, and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

### Modified Oligonucleotide

The term modified oligonucleotide describes an oligonucleotide comprising one or more sugar-modified nucleosides and/or modified internucleoside linkages. The term chimeric” oligonucleotide is a term that has been used in the literature to describe oligonucleotides with modified nucleosides.

### Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45, page 2055 and Bergstrom (2009) Current Protocols in Nucleic Acid Chemistry Suppl. 37, 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is calculated by counting the number of aligned nucleobases that are complementary (from Watson-Crick base pairing) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson-Crick base pairing is retained (e.g. 5′-methyl cytosine is considered identical to a cytosine for the purpose of calculating % identity).

The term “fully complementary” refers to 100% complementarity.

### Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned nucleobases that are identical (a match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

### Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tm) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTln(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below-10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

### Target Nucleic Acid

According to the present invention, the target nucleic acid is a nucleic acid which encodes mammalian ApoE4 and may for example be a gene, an RNA, an mRNA, a pre-mRNA, a mature mRNA or a cDNA sequence. The target may therefore be referred to as a ApoE4 target nucleic acid or APOE ε4 target nucleic acid, these terms can be used interchangeably. The oligonucleotide of the invention may, for example, target APOE ε4 pre-mRNA or mRNA.

Suitably, the target nucleic acid encodes ApoE4 protein, in particular mammalian ApoE4 protein, such as human ApoE4 protein.

In some embodiments, the target nucleic acid comprises at least residues 522 to 548 of SEQ ID NO:1 and encodes a mammalian, such as human, ApoE4 protein.

In some embodiments, the target nucleic acid comprises at least residues 516-556 of SEQ ID NO:1 and encodes a mammalian, such as human, ApoE4 protein.

In some embodiments, the target nucleic acid is SEQ ID NO: 1 or any naturally occurring variant thereof which encodes a mammalian, such as a human, ApoE4 protein. Thus, the target nucleic acid may be SEQ ID NO:1.

SEQ ID NO:2 is the human ApoE nucleic acid set forth in NCBI Reference Sequence: NM_001302690.2 (Genbank) which encodes the human ApoE3 isoform. SEQ ID NO:2 differs from SEQ ID NO:1 in residue 535 because of the rs429358 single-nucleotide polymorphism (SNP).

In some embodiments, the target nucleic acid comprises at least residues 522 to 548 of SEQ ID NO:2 with a t535c substitution and encodes a mammalian, such as human, ApoE4 protein.

In some embodiments, the target nucleic acid comprises at least residues 516-556 of SEQ ID NO:2 with a t535c substitution and encodes a mammalian, such as human, ApoE4 protein.

In some embodiments, the target nucleic acid is SEQ ID NO: 2 with a t535c substitution, or any naturally occurring variant thereof which encodes a mammalian, such as a human, ApoE4 protein. Thus, the target nucleic acid may be SEQ ID NO: 2 with a t535c substitution.

In some embodiments, the target nucleic acid encodes a cynomolgus monkey ApoE4 protein. Suitably, the target nucleic acid encoding a cynomolgus monkey ApoE4 protein comprises a sequence as shown in SEQ ID NO: 3.

In some embodiments, the target nucleic acid comprises at least residues 450-490 of SEQ ID NO:3 and encodes a mammalian, such as cynomolgous monkey, ApoE4 protein.

In some embodiments, the target nucleic acid comprises at least residues 456 to 482 of SEQ ID NO:3 and encodes a mammalian, such as cynomolgous monkey, ApoE4 protein.

In some embodiments, the target nucleic acid is SEQ ID NO: 3 or any naturally occurring variant thereof which encodes a mammalian, such as a cynomolgous monkey, ApoE4 protein. Thus, the target nucleic acid may be SEQ ID NO:3.

If employing the oligonucleotide of the invention in research or diagnostics the target nucleic acid may be a cDNA or a synthetic nucleic acid derived from DNA or RNA.

For in vivo or in vitro application, the oligonucleotide of the invention is typically capable of reducing the expression of the ApoE4 protein in a cell which is expressing the APOE &4 target nucleic acid. The contiguous sequence of nucleobases of the oligonucleotide of the invention is typically complementary to the APOE ε4 target nucleic acid, as measured across the length of the oligonucleotide, optionally with the exception of one or two mismatches, and optionally excluding nucleotide-based linker regions which may link the oligonucleotide to an optional functional group such as a conjugate, or other non-complementary terminal nucleotides (e.g. region D′or D″).

SEQ ID NOs 1-3 are presented as DNA sequences. It is understood that target RNA sequences have uracil (U) bases in place of the thymine bases (T).

The target nucleic acid is advantageously a messenger RNA, such as a mature mRNA or a pre-mRNA.

In some embodiments, the oligonucleotide of the invention targets SEQ ID NO:1.

In some embodiments, the oligonucleotide of the invention targets SEQ ID NO:2 with a t535c substitution.

In some embodiments, the oligonucleotide of the invention targets SEQ ID NO:3.

In some embodiments, the oligonucleotide of the invention targets SEQ ID NO:1 and SEQ ID NO:3.

In some embodiments, the oligonucleotide of the invention targets SEQ ID NO:1, SEQ ID NO:2 with a t535c substitution and SEQ ID NO:3.

TABLE 1
Examples of target nucleic acids

| | | | | |
|---|---|---|---|---|
| | RNA | Length | SEQ ID | |
| Species | type | (nt) | NO | Additional information |

| | | | |
|---|---|---|---|
| Human | Mature | 1 | Corresponds to NCBI |
| (homo | mRNA | | Reference Sequence: |
| sapiens) | | | NM_001302690.2 |
| | | | (Genbank) with a t535c |
| | | | substitution |
| Cynomolgous | Mature | 3 | Corresponds to NCBI |
| monkey | mRNA | | Reference Sequence: |
| (macaca | | | XM_{——}005589554.2 |
| fascicularis) | | | (Genbank) |

### Target Sequence

The term “target sequence” as used herein refers to a sequence of nucleotides present in the target nucleic acid which comprises the nucleobase sequence which is complementary to the oligonucleotide of the invention. This region of the target nucleic acid may interchangeably be referred to as the target nucleotide sequence, target sequence or target region.

In some embodiments, the target sequence consists of a region on the target nucleic acid with a nucleobase sequence that is complementary to the contiguous nucleotide sequence of the oligonucleotide of the invention. In some embodiments the target sequence is longer than the complementary sequence of a single oligonucleotide, and may, for example represent a preferred region of the target nucleic acid which may be targeted by several oligonucleotides of the invention.

The oligonucleotide of the invention comprises a contiguous nucleotide sequence which is complementary to or hybridizes to the target nucleic acid, such as a sub-sequence of the target nucleic acid, such as a target sequence described herein.

The oligonucleotide comprises a contiguous nucleotide sequence which are complementary to a target sequence present in the target nucleic acid molecule. The contiguous nucleotide sequence (and therefore the target sequence) comprises at least about 8, such as at least about 9, such as at least about 10 contiguous nucleotides, such as 8, 9, 10, 11, 12, 13, 14, 15, 16, 17, 18, 19, 20, 21, 22, 23, 24, 25, 26, 27, 28, 29 or 30 contiguous nucleotides, such as from 12-25, such as from 14-18 contiguous nucleotides.

Preferably, the target sequence is located in RNA, such as in pre-mRNA, mature mRNA or both.

The target sequence comprises the cytosine (c) residue at position 535 in SEQ ID NO:1, corresponding to the site of the rs429358 SNP.

In some embodiments, the target sequence is located in a region in the segment defined by residues 516-556 of SEQ ID NO:1.

In some embodiments, the target sequence is located in a region in the segment defined by residues 522 to 548 of SEQ ID NO:1.

In some embodiments, the target sequence comprises the cytosine (c) at position 469 in SEQ ID NO:3.

In some embodiments, the target sequence is located in a region in the segment defined by residues 450-490 of SEQ ID NO:3.

In some embodiments, the target sequence is located in a region in the segment defined by residues 456 to 482 of SEQ ID NO:3.

In some embodiments the target sequence is a sequence selected from those described in Table 2.

In some embodiments the target sequence is selected from R_25, R_40, R_46, R_66 and R_91, such as from R_25, R_40, and R_46.

In one embodiment the target sequence is R_25, corresponding to residues 522 to 535 of SEQ ID NO:1.

In one embodiment the target sequence is R_40, corresponding to residues 525 to 537 of SEQ ID NO:1.

In one embodiment the target sequence is R_46, corresponding to residues 526 to 538 of SEQ ID NO:1.

In one embodiment the target sequence is R_66, corresponding to residues 530 to 546 of SEQ ID NO:1.

In one embodiment the target sequence is R_91, corresponding to residues 535 to 548 of SEQ ID NO:1.

It is to be understood that target RNA sequence regions have uracil (U) bases in place of any thymine (T) bases.

TABLE 2
Examples of target sequences on SEQ ID NO: 1

Position on SEQ ID NO: 1

| Ref | Start | Stop |
|---|---|---|
| R_4 | 516 | 535 |
| R_5 | 517 | 536 |
| R_6 | 517 | 535 |
| R_7 | 518 | 538 |
| R_8 | 518 | 537 |
| R_9 | 518 | 536 |
| R_10 | 518 | 535 |
| R_11 | 519 | 538 |
| R_12 | 519 | 537 |
| R_13 | 519 | 536 |
| R_14 | 519 | 535 |
| R_15 | 520 | 539 |
| R_16 | 520 | 538 |
| R_17 | 520 | 537 |
| R_18 | 520 | 536 |
| R_19 | 520 | 535 |
| R_20 | 521 | 540 |
| R_21 | 521 | 539 |
| R_22 | 521 | 538 |
| R_23 | 521 | 537 |
| R_24 | 521 | 536 |
| R_25 | 522 | 535 |
| R_26 | 522 | 541 |
| R_27 | 522 | 540 |
| R_28 | 522 | 539 |
| R_29 | 522 | 537 |
| R_30 | 523 | 542 |
| R_31 | 523 | 541 |
| R_32 | 523 | 540 |
| R_33 | 523 | 539 |
| R_34 | 523 | 538 |
| R_35 | 524 | 543 |
| R_36 | 524 | 542 |
| R_37 | 524 | 541 |
| R_38 | 524 | 540 |
| R_39 | 524 | 539 |
| R_40 | 525 | 537 |
| R_41 | 525 | 544 |
| R_42 | 525 | 543 |
| R_43 | 525 | 542 |
| R_44 | 525 | 541 |
| R_45 | 525 | 540 |
| R_46 | 526 | 538 |
| R_47 | 526 | 545 |
| R_48 | 526 | 544 |
| R_49 | 526 | 543 |
| R_50 | 526 | 541 |
| R_51 | 527 | 546 |
| R_52 | 527 | 545 |
| R_53 | 527 | 544 |
| R_54 | 527 | 543 |
| R_55 | 527 | 542 |
| R_56 | 528 | 547 |
| R_57 | 528 | 546 |
| R_58 | 528 | 545 |
| R_59 | 528 | 544 |
| R_60 | 528 | 543 |
| R_61 | 529 | 548 |
| R_62 | 529 | 547 |
| R_63 | 529 | 546 |
| R_64 | 529 | 545 |
| R_65 | 529 | 544 |
| R_66 | 530 | 546 |
| R_67 | 530 | 549 |
| R_68 | 530 | 548 |
| R_69 | 530 | 547 |
| R_70 | 530 | 545 |
| R_71 | 531 | 550 |
| R_72 | 531 | 549 |
| R_73 | 531 | 548 |
| R_74 | 531 | 547 |
| R_75 | 531 | 546 |
| R_76 | 532 | 551 |
| R_77 | 532 | 550 |
| R_78 | 532 | 549 |
| R_79 | 532 | 548 |
| R_80 | 532 | 547 |
| R_81 | 533 | 552 |
| R_82 | 533 | 551 |
| R_83 | 533 | 550 |
| R_84 | 533 | 549 |
| R_85 | 533 | 548 |
| R_86 | 534 | 553 |
| R_87 | 534 | 552 |
| R_88 | 534 | 551 |
| R_89 | 534 | 550 |
| R_90 | 534 | 549 |
| R_91 | 535 | 548 |
| R_92 | 535 | 554 |
| R_93 | 535 | 553 |
| R_94 | 535 | 552 |
| R_95 | 535 | 551 |
| R_96 | 535 | 550 |

### Target Cell

The term a “target cell” as used herein refers to a cell which is expressing the target nucleic acid. In some embodiments the target cell may be in vivo or in vitro. In some embodiments the target cell is a mammalian cell such as a rodent cell, such as a mouse cell or a rat cell, or a primate cell such as a monkey cell (e.g. a cynomolgus monkey cell) or a human cell.

In preferred embodiments the target cell expresses human ApoE4 mRNA, such as ApoE4 pre-mRNA, e.g. SEQ ID NO:1, or ApoE4 mature mRNA. In some embodiments the target cell expresses cynomolgous monkey ApoE4 mRNA, such as ApoE4 mature mRNA, e.g. SEQ ID NO:3, Any poly-A tail of ApoE4 mRNA is typically disregarded for antisense oligonucleotide targeting.

### Naturally Occurring Variant

The term “naturally occurring variant” refers to variants of APOE ε4 gene or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms (SNPs) other than the rs429358 SNP (including silent SNPs), and allelic variants. Based on the presence of the sufficient complementary sequence to the oligonucleotide, the oligonucleotide of the invention may therefore target the target nucleic acid and naturally occurring variants thereof.

In some embodiments, the naturally occurring variants have at least 95% such as at least 98% or at least 99% homology to a mammalian ApoE4 target nucleic acid, such as a target nucleic acid selected form the group consisting of SEQ ID NO:1 and 3. In some embodiments the naturally occurring variants have at least 99% homology to the human APOE ε4 target nucleic acid of SEQ ID NO: 1.

### Modulation of Expression

The term “modulation of expression” as used herein is to be understood as an overall term for an oligonucleotide's ability to alter the amount of ApoE4 protein or ApoE4 mRNA as compared to the amount of ApoE4 protein or ApoE4 mRNA before administration of the oligonucleotide. Alternatively, modulation of expression may be determined by reference to a control experiment. It is generally understood that the control is an individual or target cell treated with a saline composition or an individual or target cell treated with a non-targeting oligonucleotide (mock).

One type of modulation is the ability of an oligonucleotide to inhibit, down-regulate, reduce, suppress, remove, stop, block, prevent, lessen, lower, avoid or terminate expression of ApoE4, e.g. by degradation of ApoE4 mRNA or blockage of transcription.

### High-Affinity Modified Nucleosides

A high affinity modified nucleoside is a modified nucleotide which, when incorporated into the oligonucleotide enhances the affinity of the oligonucleotide for its complementary target, for example as measured by the melting temperature (T^{m}). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

### Sugar Modifications

The oligomer of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (Locked Nucleic Acid, or “LNA”), or an unlinked ribose ring which typically lacks a bond between the C2 and C3 carbons (e.g. Unlocked Nucleic Acid, or “UNA”). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

### 2′ Sugar Modified Nucleosides

A 2′ sugar modified nucleoside is a nucleoside which has a substituent other than H or —OH at the 2′ position (2′ substituted nucleoside) or comprises a 2′-linked biradical capable of forming a bridge between the 2′ carbon and a second carbon in the ribose ring, such as LNA (2′-4′ biradical-bridged) nucleosides.

Indeed, much focus has been spent on developing 2′ sugar substituted nucleosides, and numerous 2′-substituted nucleosides have been found to have beneficial properties when incorporated into oligonucleotides. For example, the 2′ modified sugar may provide enhanced binding affinity and/or increased nuclease resistance to the oligonucleotide. Examples of 2′-substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

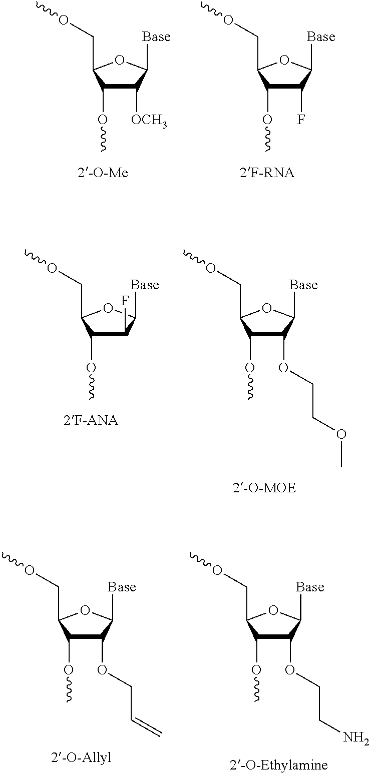

### Locked Nucleic Acid Nucleosides (LNA Nucleoside)

A “LNA nucleoside” is a 2′-sugar modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, Mitsuoka et al., Nucleic Acids Research 2009, 37(4), 1225-1238, and Wan and Seth, J. Medical Chemistry 2016, 59, 9645-9667.

Further non-limiting, exemplary LNA nucleosides are disclosed in Scheme 1.

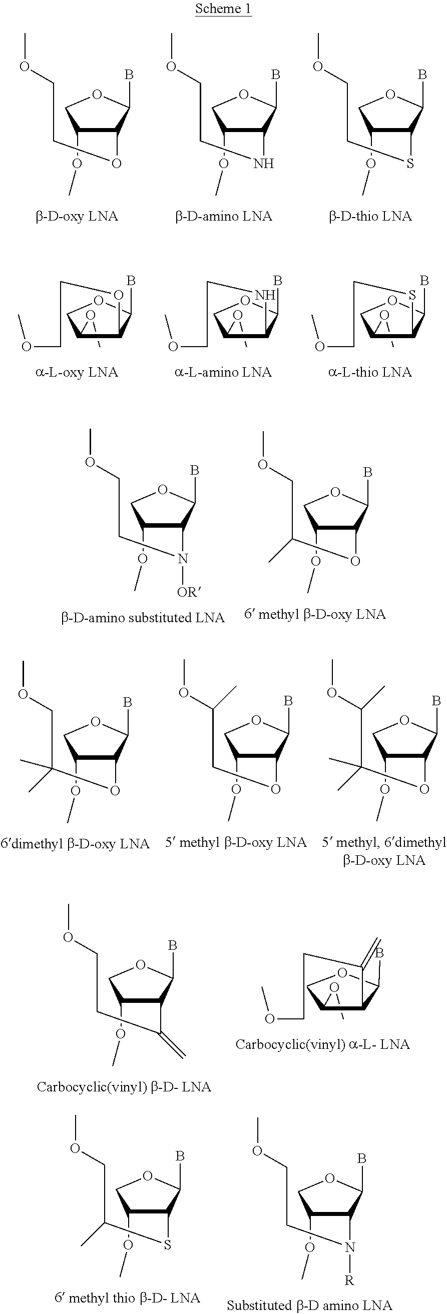

Particular LNA nucleosides are beta-D-oxy-LNA, 6′-methyl-beta-D-oxy LNA such as (R) or (S)-6′-methyl-beta-D-oxy-LNA (ScET) and ENA. A particularly advantageous LNA is beta-D-oxy-LNA.

### Chemical Group Definitions

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C₁-C₈ alkyl groups are methyl, ethyl, propyl, isopropyl, butyl, isobutyl, tert.-butyl, the isomeric pentyls, the isomeric hexyls, the isomeric heptyls and the isomeric octyls, particularly methyl, ethyl, propyl, butyl and pentyl. Particular examples of alkyl are methyl, ethyl and propyl.

The term “cycloalkyl”, alone or in combination, signifies a cycloalkyl ring with 3 to 8 carbon atoms and particularly a cycloalkyl ring with 3 to 6 carbon atoms. Examples of cycloalkyl are cyclopropyl, cyclobutyl, cyclopentyl, cyclohexyl, cycloheptyl and cyclooctyl, more particularly cyclopropyl and cyclobutyl. A particular example of “cycloalkyl” is cyclopropyl.

The term “alkoxy”, alone or in combination, signifies a group of the formula alkyl-O—in which the term “alkyl” has the previously given significance, such as methoxy, ethoxy, n-propoxy, isopropoxy, n-butoxy, isobutoxy, sec.butoxy and tert.butoxy. Particular “alkoxy” are methoxy and ethoxy. Methoxyethoxy is a particular example of “alkoxyalkoxy”.

The term “oxy”, alone or in combination, signifies the —O-group.

The term “alkenyl”, alone or in combination, signifies a straight-chain or branched hydrocarbon residue comprising an olefinic bond and up to 8, preferably up to 6, particularly preferred up to 4 carbon atoms. Examples of alkenyl groups are ethenyl, 1-propenyl, 2-propenyl, isopropenyl, 1-butenyl, 2-butenyl, 3-butenyl and isobutenyl.

The term “alkynyl”, alone or in combination, signifies a straight-chain or branched hydrocarbon residue comprising a triple bond and up to 8, preferably up to 6, particularly preferred up to 4 carbon atoms.

The terms “halogen” or “halo”, alone or in combination, signifies fluorine, chlorine, bromine or iodine and particularly fluorine, chlorine or bromine, more particularly fluorine. The term “halo”, in combination with another group, denotes the substitution of said group with at least one halogen, particularly substituted with one to five halogens, particularly one to four halogens, i.e. one, two, three or four halogens.

The term “haloalkyl”, alone or in combination, denotes an alkyl group substituted with at least one halogen, particularly substituted with one to five halogens, particularly one to three halogens. Examples of haloalkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl or trifluoromethyl. Fluoromethyl, difluoromethyl and trifluoromethyl are particular “haloalkyl”.

The term “halocycloalkyl”, alone or in combination, denotes a cycloalkyl group as defined above substituted with at least one halogen, particularly substituted with one to five halogens, particularly one to three halogens. Particular example of “halocycloalkyl” are halocyclopropyl, in particular fluorocyclopropyl, difluorocyclopropyl and trifluorocyclopropyl.

The terms “hydroxyl” and “hydroxy”, alone or in combination, signify the —OH group.

The terms “thiohydroxyl” and “thiohydroxy”, alone or in combination, signify the —SH group.

The term “carbonyl”, alone or in combination, signifies the —C(O)-group.

The term “carboxy” or “carboxyl”, alone or in combination, signifies the —COOH group.

The term “amino”, alone or in combination, signifies the primary amino group (—NH₂), the secondary amino group (—NH—), or the tertiary amino group (—N—).

The term “alkylamino”, alone or in combination, signifies an amino group as defined above substituted with one or two alkyl groups as defined above.

The term “sulfonyl”, alone or in combination, means the —SO₂ group.

The term “sulfinyl”, alone or in combination, signifies the —SO-group.

The term “sulfanyl”, alone or in combination, signifies the —S-group.

The term “cyano”, alone or in combination, signifies the —CN group.

The term “azido”, alone or in combination, signifies the —N₃ group.

The term “nitro”, alone or in combination, signifies the NO₂ group.

The term “formyl”, alone or in combination, signifies the —C(O)H group.

The term “carbamoyl”, alone or in combination, signifies the —C(O)NH₂ group.

The term “cabamido”, alone or in combination, signifies the —NH—C(O)—NH₂ group.

The term “aryl”, alone or in combination, denotes a monovalent aromatic carbocyclic mono-or bicyclic ring system comprising 6 to 10 carbon ring atoms, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples of aryl include phenyl and naphthyl, in particular phenyl.

The term “heteroaryl”, alone or in combination, denotes a monovalent aromatic heterocyclic mono- or bicyclic ring system of 5 to 12 ring atoms, comprising 1, 2, 3 or 4 heteroatoms selected from N, O and S, the remaining ring atoms being carbon, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples of heteroaryl include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, azepinyl, diazepinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, quinoxalinyl, carbazolyl or acridinyl.

The term “heterocyclyl”, alone or in combination, signifies a monovalent saturated or partly unsaturated mono-or bicyclic ring system of 4 to 12, in particular 4 to 9 ring atoms, comprising 1, 2, 3 or 4 ring heteroatoms selected from N, O and S, the remaining ring atoms being carbon, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples for monocyclic saturated heterocyclyl are azetidinyl, pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Examples for bicyclic saturated heterocycloalkyl are 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, or 3-thia-9-aza-bicyclo[3.3.1 ]nonyl. Examples for partly unsaturated heterocycloalkyl are dihydrofuryl, imidazolinyl, dihydro-oxazolyl, tetrahydro-pyridinyl or dihydropyranyl.

### Pharmaceutically Acceptable Salts

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. These salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of compounds of formula (I) are the salts of hydrochloric acid, hydrobromic acid, sulfuric acid, phosphoric acid and methanesulfonic acid.

### Protecting Group

The term “protecting group”, alone or in combination, signifies a group which selectively blocks a reactive site in a multifunctional compound such that a chemical reaction can be carried out selectively at another unprotected reactive site. Protecting groups can be removed. Exemplary protecting groups are amino-protecting groups, carboxy-protecting groups or hydroxy-protecting groups.

### Nuclease Mediated Degradation

Nuclease mediated degradation refers to an oligonucleotide capable of mediating degradation of a complementary nucleotide sequence when forming a duplex with such a sequence.

In some embodiments, the oligonucleotide may function via nuclease mediated degradation of the target nucleic acid, where the oligonucleotides of the invention are capable of recruiting a nuclease, particularly and endonuclease, preferably endoribonuclease (RNase), such as RNase H. Examples of oligonucleotide designs which operate via nuclease mediated mechanisms are oligonucleotides which typically comprise a region of at least 5 or 6 consecutive DNA nucleosides and are flanked on one side or both sides by affinity enhancing nucleosides, for example gapmers, headmers and tailmers.

### RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference). For use in determining RHase H activity, recombinant human RNase H1 is available from Lubio Science GmbH, Lucerne, Switzerland.

### Gapmer

The antisense oligonucleotide of the invention, or contiguous nucleotide sequence thereof, may be a gapmer, also termed gapmer oligonucleotide or gapmer designs. The antisense gapmers are commonly used to inhibit a target nucleic acid via RNase H mediated degradation. A gapmer oligonucleotide comprises at least three distinct structural regions a 5′-flank, a gap and a 3′-flank, F-G-F′ in the ‘5 to 3’ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides and are positioned adjacent to a sugar modified nucleoside of the 5′ (F) or 3′ (F′) region respectively. The flanks may further be defined by having at least one sugar modified nucleoside at the end most distant from the gap region, i.e. at the 5′ end of the 5′ flank and at the 3′ end of the 3′ flank.

Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 32 nucleosides, such as 13 to 24, such as 14 to 22 nucleosides, such as from 14 to 17, such as 16 to 18 nucleosides, such as 16 to 20 nuclotides.

By way of example, the gapmer oligonucleotide of the present invention can be represented by the following formulae:

F₁₋₈-G₅₋₁₆-F′₁₋₈, such as

F₁₋₈-G₇₋₁₆-F′₂₋₈, such as

F₃₋₈-G₆₋₁₄-F′₂₋₈

with the proviso that the overall length of the gapmer regions F-G-F′ is at least 10, such as at least 12, such as at least 14 nucleotides in length.

In an aspect of the invention the antisense oligonucleotide or contiguous nucleotide sequence thereof consists of or comprises a gapmer of formula 5′-F-G-F′-3′, where region F and F′ independently comprise or consist of 1-8 nucleosides, of which 2-4 are 2′ sugar modified and defines the 5′ and 3′ end of the F and F′ region, and G is a region between 6 and 16 nucleosides which are capable of recruiting RNaseH.

Regions F, G and F′ are further defined below and can be incorporated into the F-G-F′ formula.

### Gapmer-Region G

Region G (gap region) of the gapmer is a region of nucleosides which enables the oligonucleotide to recruit RNaseH, such as human RNase H1, typically DNA nucleosides. RNaseH is a cellular enzyme which recognizes the duplex between DNA and RNA, and enzymatically cleaves the RNA molecule. Suitably gapmers may have a gap region (G) of at least 5 or 6 contiguous DNA nucleosides, such as 5-16 contiguous DNA nucleosides, such as 6-15 contiguous DNA nucleosides, such as 7-14 contiguous DNA nucleosides, such as 8-12 contiguous DNA nucleotides, such as 8-12 contiguous DNA nucleotides in length. The gap region G may, in some embodiments consist of 6, 7, 8, 9, 10, 11, 12, 13, 14, 15 or 16 contiguous DNA nucleosides. One or more cytosine (c) DNA in the gap region may in some instances be methylated (e.g., when a DNA c is followed by a DNA g), and such 5′-methyl-cytosine residues can be annotated as meC or with an e instead of a c. 5′-substituted DNA nucleosides, such as 5′ methyl DNA nucleosides, have been reported for use in DNA gap regions (EP 2 742 136).

In some embodiments the gap region G may consist of 6, 7, 8, 9, 10, 11, 12, 13, 14, 15 or 16 contiguous phosphorothioate linked DNA nucleosides. In some embodiments, all internucleoside linkages in the gap are phosphorothioate linkages.

Whilst traditional gapmers have a DNA gap region, there are numerous examples of modified nucleosides which allow for RNaseH recruitment when they are used within the gap region. Modified nucleosides which have been reported as being capable of recruiting RNaseH when included within a gap region include, for example, alpha-L-LNA, C4′ alkylated DNA (as described in PCT/EP2009/050349 and Vester et al., Bioorg. Med. Chem. Lett. 18 (2008) 2296 -2300, both incorporated herein by reference), arabinose derived nucleosides like ANA and 2′F-ANA (Mangos et al. 2003 J. AM. CHEM. SOC. 125, 654-661), UNA (unlocked nucleic acid) (as described in Fluiter et al., Mol. Biosyst., 2009, 10, 1039 incorporated herein by reference). UNA is unlocked nucleic acid, typically where the bond between C2 and C3 of the ribose has been removed, forming an unlocked “sugar” residue. The modified nucleosides used in such gapmers may be nucleosides which adopt a 2′ endo (DNA like) structure when introduced into the gap region, i.e. modifications which allow for RNaseH recruitment). In some embodiments the DNA Gap region (G) described herein may optionally contain 1 to 3 sugar modified nucleosides which adopt a 2′ endo (DNA like) structure when introduced into the gap region.

### Region G—“Gap-breaker”

Alternatively, there are numerous reports of the insertion of a modified nucleoside which confers a 3′ endo conformation into the gap region of gapmers, whilst retaining some RNaseH activity. Such gapmers with a gap region comprising one or more 3′endo modified nucleosides are referred to as “gap-breaker” or “gap-disrupted” gapmers, see for example WO2013/022984. Gap-breaker oligonucleotides retain sufficient region of DNA nucleosides within the gap region to allow for RNaseH recruitment. The ability of gapbreaker oligonucleotide design to recruit RNaseH is typically sequence or even compound specific—see Rukov et al. 2015 Nucl. Acids Res. Vol. 43 pp. 8476-8487, which discloses “gapbreaker” oligonucleotides which recruit RNaseH which in some instances provide a more specific cleavage of the target RNA. Modified nucleosides used within the gap region of gap-breaker oligonucleotides may for example be modified nucleosides which confer a 3′endo confirmation, such 2′-O-methyl (OMe) or 2′-O-MOE (MOE) nucleosides, or beta-D LNA nucleosides (the bridge between C2′ and C4′ of the ribose sugar ring of a nucleoside is in the beta conformation), such as beta-D-oxy LNA or ScET nucleosides.

As with gapmers containing region G described above, the gap region of gap-breaker or gap-disrupted gapmers, have a DNA nucleoside at the 5′ end of the gap (adjacent to the 3′ nucleoside of region F), and a DNA nucleoside at the 3′ end of the gap (adjacent to the 5′ nucleoside of region F′). Gapmers which comprise a disrupted gap typically retain a region of at least 3 or 4 contiguous DNA nucleosides at either the 5′ end or 3′ end of the gap region.

Exemplary designs for gap-breaker oligonucleotides include

F₁₋₈−[D₃₋₄−E₁−D₃₋₄]−F′₁₋₈

F₁₋₈−[D₁₋₄−D₃₋₄]−F′₁₋₈

F₁₋₈−[D₃₋₄−E₁−D₁₋₄]−F′₁₋₈

wherein region G is within the brackets [Dn-Er-Dm], D is a contiguous sequence of DNA nucleosides, E is a modified nucleoside (the gap-breaker or gap-disrupting nucleoside), and F and F′ are the flanking regions as defined herein, and with the proviso that the overall length of the gapmer regions F-G-F′ is at least 12, such as at least 14 nucleotides in length.

In some embodiments, region G of a gap disrupted gapmer comprises at least 6 DNA nucleosides, such as 6, 7, 8, 9, 10, 11, 12, 13, 14, 15 or 16 DNA nucleosides. As described above, the DNA nucleosides may be contiguous or may optionally be interspersed with one or more modified nucleosides, with the proviso that the gap region G is capable of mediating RNaseH recruitment.

### Gapmer-Flanking Regions, F and F′

Region F is positioned immediately adjacent to the 5′ DNA nucleoside of region G. The 3′ most nucleoside of region F is a sugar modified nucleoside, such as a high affinity sugar modified nucleoside, for example a 2′ substituted nucleoside, such as a MOE nucleoside, or an LNA nucleoside.

Region F′ is positioned immediately adjacent to the 3′ DNA nucleoside of region G. The 5′ most nucleoside of region F′ is a sugar modified nucleoside, such as a high affinity sugar modified nucleoside, for example a 2′ substituted nucleoside, such as a MOE nucleoside, or an LNA nucleoside.

Region F is 1-8 contiguous nucleotides in length, such as 2-6, such as 3-4 contiguous nucleotides in length. Advantageously the 5′ most nucleoside of region F is a sugar modified nucleoside. In some embodiments the two 5′ most nucleoside of region F are sugar modified nucleoside. In some embodiments the 5′ most nucleoside of region F is an LNA nucleoside. In some embodiments the two 5′ most nucleoside of region F are LNA nucleosides. In some embodiments the two 5′ most nucleoside of region F are 2′ substituted nucleoside nucleosides, such as two 3′ MOE nucleosides. In some embodiments the 5′ most nucleoside of region F is a 2′ substituted nucleoside, such as a MOE nucleoside.

Region F′ is 1-8 contiguous nucleotides in length, such as 2-8, such as 3-6, such as 4-5 contiguous nucleotides in length. Advantageously, embodiments the 3′ most nucleoside of region F′ is a sugar modified nucleoside. In some embodiments the two 3′ most nucleoside of region F′ are sugar modified nucleoside. In some embodiments the two 3′ most nucleoside of region F′ are LNA nucleosides. In some embodiments the 3′ most nucleoside of region F′ is an LNA nucleoside. In some embodiments the two 3′ most nucleoside of region F′ are 2′ substituted nucleoside nucleosides, such as two 3′ MOE nucleosides. In some embodiments the 3′ most nucleoside of region F′ is a 2′ substituted nucleoside, such as a MOE nucleoside.

It should be noted that when the length of region F or F′ is one, it is advantageously an LNA nucleoside.

In some embodiments, region F and F′ independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, region F and F′ independently comprises both LNA and a 2′ substituted modified nucleosides (mixed wing design).

In some embodiments, region F and F′ consists of only one type of sugar modified nucleosides, such as only MOE or only beta-D-oxy LNA or only ScET. Such designs are also termed uniform flanks or uniform gapmer design.

In some embodiments, all the nucleosides of region F or F′, or F and F′ are LNA nucleosides, such as independently selected from beta-D-oxy LNA, ENA or ScET nucleosides. In some embodiments region F consists of 1-5, such as 2-4, such as 3-4 such as 1, 2, 3, 4 or 5 contiguous LNA nucleosides. In some embodiments, all the nucleosides of region F and F′ are beta-D-oxy LNA nucleosides.

In some embodiments, all the nucleosides of region F or F′, or F and F′ are 2′ substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′ substituted nucleosides, such as OMe or MOE nucleosides. In some embodiments it is the 5′ (F) flanking region that consists 2′ substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists 2′ substituted nucleosides, such as OMe or MOE nucleosides whereas the 5′ (F) flanking region comprises at least one LNA nucleoside, such as beta-D-oxy LNA nucleosides or cET nucleosides.

In some embodiments, all the modified nucleosides of region F and F′ are LNA nucleosides, such as independently selected from beta-D-oxy LNA, ENA or ScET nucleosides, wherein region F or F′, or F and F′ may optionally comprise DNA nucleosides (an alternating flank, see definition of these for more details). In some embodiments, all the modified nucleosides of region F and F′ are beta-D-oxy LNA nucleosides, wherein region F or F′, or F and F′ may optionally comprise DNA nucleosides (an alternating flank, see definition of these for more details).

In some embodiments the 5′ most and the 3′ most nucleosides of region F and F′ are LNA nucleosides, such as beta-D-oxy LNA nucleosides or ScET nucleosides.

In some embodiments, the internucleoside linkage between region F and region G is a phosphorothioate internucleoside linkage. In some embodiments, the internucleoside linkage between region F′ and region G is a phosphorothioate internucleoside linkage. In some embodiments, the internucleoside linkages between the nucleosides of region F or F′, F and F′ are phosphorothioate internucleoside linkages.

### LNA Gapmer

An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments the LNA gapmer is of formula: [LNA]₁₋₅-[region G]-[LNA]₁₋₅, wherein region G is as defined in the Gapmer region G definition.

In one embodiment the LNA gapmer is of the formula [LNA]₄-[region G]₁₀₋₁₂-[LNA]₄

### MOE Gapmers

A MOE gapmers is a gapmer wherein regions F and F′ consist of MOE nucleosides. In some embodiments the MOE gapmer is of design [MOE]₁₋₈-[Region G]₅₋₁₆-[MOE]₁₋₈, such as [MOE]₂₋₇-[Region G]₆₋₁₄-[MOE]₂₋₇, such as [MOE]₃₋₆-[Region G]₈₋₁₂-[MOE]₃₋₆, wherein region G is as defined in the Gapmer definition. MOE gapmers with a 5-10-5 design (MOE-DNA-MOE) have been widely used in the art.

### Mixed Wing Gapmer

A mixed wing gapmer is an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as MOE nucleosides. In some embodiments wherein at least one of region F and F′, or both region F and F′ comprise at least one LNA nucleoside, the remaining nucleosides of region F and F′ are independently selected from the group consisting of MOE and LNA. In some embodiments wherein at least one of region F and F′, or both region F and F′ comprise at least two LNA nucleosides, the remaining nucleosides of region F and F′ are independently selected from the group consisting of MOE and LNA. In some mixed wing embodiments, one or both of region F and F′ may further comprise one or more DNA nucleosides.

Mixed wing gapmer designs are disclosed in WO2008/049085 and WO2012/109395, both of which are hereby incorporated by reference.

### Alternating Flank Gapmers

Flanking regions may comprise both LNA and DNA nucleoside and are referred to as “alternating flanks” as they comprise an alternating motif of LNA-DNA-LNA nucleosides. Gapmers comprising such alternating flanks are referred to as “alternating flank gapmers”. “Alternating flank gapmers” are LNA gapmer oligonucleotides where at least one of the flanks (F or F′) comprises DNA in addition to the LNA nucleoside(s). In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F and/or F′ region are LNA nucleosides.

Alternating flank LNA gapmers are disclosed in WO2016/127002.

An alternating flank region may comprise up to 3 contiguous DNA nucleosides, such as 1 to 2 or 1 or 2 or 3 contiguous DNA nucleosides.

The alternating flank can be annotated as a series of integers, representing a number of LNA nucleosides (L) followed by a number of DNA nucleosides (D), for example

[L]₁₋₃−[D]₁₋₄−[L]₁₋₃

[L]₁₋₂−[D]₁₋₂−[L]₁₋₂−[D]₁₋₂−[L]₁₋₂

In oligonucleotide designs these will often be represented as numbers such that 2-2-1 represents 5′ [L]₂-[D]₂-[L] 3′, and 1-1-1-1-1 represents 5′ [L]-[D]-[L]-[D]-[L] 3′. The length of the flank (region F and F′) in oligonucleotides with alternating flanks may independently be 3 to 10 nucleosides, such as 4 to 8, such as 5 to 6 nucleosides, such as 4, 5, 6 or 7 modified nucleosides. In some embodiments only one of the flanks in the gapmer oligonucleotide is alternating while the other is constituted of LNA nucleotides. It may be advantageous to have at least two LNA nucleosides at the 3′ end of the 3′ flank (F′), to confer additional exonuclease resistance. In one embodiment the flanks in the alternating flank gapmer have an overall length from 5- to 8 nucleosides of which 3 to 5 are LNA nucleosides. Some examples of oligonucleotides with alternating flanks are:

[L]₁₋₅−[D]₁₋₄−[L]₁₋₃−[G]₅₋₁₆−[L]₂₋₆

[L]₁₋₂−[D]₂₋₃−[L]₃₋₄−[G]₅₋₇−[L]₁₋₂−[D]₂₋₃−[L]₂₋₃

[L]₁₋₂−[D]₁₋₂−[L]₁₋₂−[D]₁₋₂−[L]₁₋₂−[G]₅₋₁₆−[L]₁₋₂−[D]₁₋₃−[L]₂₋₄

[L]₁₋₅−[G]₅₋₁₆−[L]−[D]−[L]−[D]−[L]₂

[L]₄−[G]₆₋₁₀−[L]−[D]−[D]₃−[L]₂

with the proviso that the overall length of the gapmer is at least 12, such as at least 14 nucleotides in length.

### Region D′ or D″ in an Oligonucleotide

The oligonucleotide of the invention may in some embodiments comprise or consist of the contiguous nucleotide sequence of the oligonucleotide which is complementary to the target nucleic acid, such as the gapmer F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively, it may be used to provide exonucleoase protection or for ease of synthesis or manufacture.

Region D′ and D″ can be attached to the 5′ end of region F or the 3′ end of region F′, respectively to generate designs of the following formulas D′-F-G-F′, F-G-F′-D″ or D′-F-G-F′-D″. In this instance the F-G-F′ is the gapmer portion of the oligonucleotide and region D′or D″ constitute a separate part of the oligonucleotide.

Region D′or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs (e.g. gapmer regions) within a single oligonucleotide.

In one embodiment the oligonucleotide of the invention comprises a region D′ and/or D″ in addition to the contiguous nucleotide sequence which constitutes the gapmer.

In some embodiments, the oligonucleotide of the present invention can be represented by the following formulae:

F-G-F′; in particular F₂₋₈-G₆₋₁₆-F′₂₋₈

D′-F-G-F′, in particular D′₂₋₃-F₁₋₈-G₆₋₁₆-F′₂₋₈

F-G-F′-D″, in particular F₂₋₈-G₆₋₁₆-F′₂₋₈-D″₁₋₃

D′-F-G-F′-D″, in particular D′₁₋₃-F₂₋₈-G₆₋₁₆-F′₂₋₈-D″₁₋₃

In some embodiments the internucleoside linkage positioned between region D′ and region F is a phosphodiester linkage. In some embodiments the internucleoside linkage positioned between region F′ and region D″ is a phosphodiester linkage.

### Conjugate

The term conjugate as used herein refers to an oligonucleotide which is covalently linked to a non-nucleotide moiety (conjugate moiety or region C or third region).

Conjugation of the oligonucleotide of the invention to one or more non-nucleotide moieties may improve the pharmacology of the oligonucleotide, e.g. by affecting the activity, cellular distribution, cellular uptake or stability of the oligonucleotide. In some embodiments the conjugate moiety modify or enhance the pharmacokinetic properties of the oligonucleotide by improving cellular distribution, bioavailability, metabolism, excretion, permeability, and/or cellular uptake of the oligonucleotide. In particular, the conjugate may target the oligonucleotide to a specific organ, tissue or cell type and thereby enhance the effectiveness of the oligonucleotide in that organ, tissue or cell type. At the same time the conjugate may serve to reduce activity of the oligonucleotide in non-target cell types, tissues or organs, e.g. off target activity or activity in non-target cell types, tissues or organs.

Oligonucleotide conjugates and their synthesis has also been reported in comprehensive reviews by Manoharan in Antisense Drug Technology, Principles, Strategies, and Applications, S.T. Crooke, ed., Ch. 16, Marcel Dekker, Inc., 2001 and Manoharan, Antisense and Nucleic Acid Drug Development, 2002, 12, 103, each of which is incorporated herein by reference in its entirety.

In an embodiment, the non-nucleotide moiety (conjugate moiety) is selected from the group consisting of carbohydrates (e.g. GalNAc), cell surface receptor ligands, drug substances, hormones, lipophilic substances, polymers, proteins, peptides, toxins (e.g. bacterial toxins), vitamins, viral proteins (e.g. capsids) or combinations thereof.

In some embodiments, the conjugate is an antibody or an antibody fragment which has a specific affinity for a transferrin receptor, for example as disclosed in WO 2012/143379 herby incorporated by reference. In some embodiments the non-nucleotide moiety is an antibody or antibody fragment, such as an antibody or antibody fragment that facilitates delivery across the blood-brain-barrier, in particular an antibody or antibody fragment targeting the transferrin receptor.

### Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence complementary to the target nucleic acid (region A).

In some embodiments of the invention the conjugate or oligonucleotide conjugate of the invention may optionally, comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In a preferred embodiment the nuclease susceptible linker comprises between 1 and 10 nucleosides, such as 1, 2, 3, 4, 5, 6, 7, 8, 9 or 10 nucleosides, more preferably between 2 and 6 nucleosides and most preferably between 2 and 4 linked nucleosides comprising at least two consecutive phosphodiester linkages, such as at least 3 or 4 or 5 consecutive phosphodiester linkages. Preferably the nucleosides are DNA or RNA. Phosphodiester containing biocleavable linkers are described in more detail in WO 2014/076195 (hereby incorporated by reference).

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups The oligonucleotide conjugates of the present invention can be constructed of the following regional elements A-C, A-B-C, A-B-Y-C, A-Y-B-C or A-Y-C. In some embodiments the linker (region Y) is an amino alkyl, such as a C2-C36 amino alkyl group, including, for example C6 to C12 amino alkyl groups. In a preferred embodiment the linker (region Y) is a C6 amino alkyl group.

### Treatment

The term ‘treatment’ as used herein refers to both treatment of an existing disease (e.g. a disease, disorder or dysfunction as herein referred to), or prevention of a disease, i.e. prophylaxis. It will therefore be recognized that treatment as referred to herein may, in some embodiments, be prophylactic.

### DETAILED DESCRIPTION OF THE INVENTION

### The Oligonucleotides of the Invention

The invention relates to oligonucleotides capable of modulating expression of ApoE4, such as reducing (down-regulating) ApoE4 expression. The modulation is achieved by hybridizing to a target nucleic acid encoding ApoE4. The target nucleic acid may be a mammalian APOE ε4 mRNA sequence, such as a sequence selected from the group consisting of SEQ ID NOS: 1 and 3.

The oligonucleotide is an antisense oligonucleotide which targets APOE ε4 nucleic acid, resulting in reduced ApoE4 expression.

Advantageously, the oligonucleotide sequence is complementary to a target sequence in SEQ ID NO:1 which comprises residue 535 of SEQ ID NO:1. Preferably, the nucleotide of the contiguous nucleotide sequence which is complementary to the nucleotide at position 535 of SEQ ID NO:1 comprises a guanine (g) nucleobase, or a modified nucleobase thereof which allows Watson-Crick base pairing with a cytosine (c) at position 535 of SEQ ID NO:1.

Advantageously, the antisense oligonucleotide is capable of modulating the expression of the target by reducing or down-regulating it. Preferably, such modulation produces an inhibition of expression of at least 20% compared to the normal expression level of the target, more preferably at least 30%, at least 40%, at least 50%, at least 60%, at least 70%, at least 80%, or at least 90% inhibition compared to the normal expression level of the target. Also preferred is that the antisense oligonucleotide is capable of reducing the expression level of the target by at least 20% compared to the normal expression level of the target, more preferably at least 30%, at least 40%, at least 50%, at least 60%, at least 70%, at least 80%, at least 90%, or 100% inhibition compared to the normal expression level of the target.

In some embodiments, the antisense oligonucleotide is capable of reducing expression levels of ApoE4 mRNA by at least 60% or 70% in vitro following application of 25 μM oligonucleotide to neuroblastoma cells comprising at least one copy of APOE ε4 in the genome, as compared to the normal expression level of the ApoE4 mRNA. In some embodiments, the antisense oligonucleotide is capable of reducing expression levels of ApoE4 mRNA by at least 50% or 60% in vitro following application of 5 μM oligonucleotide to neuroblastoma cells comprising at least one copy of APOE ε4 in the genome, as compared to the normal expression level of the ApoE4 mRNA. Suitably, the examples provide an assay which may be used to measure ApoE4 RNA inhibition (Example 1).

The target modulation is triggered by the hybridization between a contiguous nucleotide sequence of the oligonucleotide and the target nucleic acid. In some embodiments the oligonucleotide comprises mismatches between the oligonucleotide and the target nucleic acid. Despite mismatches hybridization to the target nucleic acid may still be sufficient to show a desired modulation of ApoE4 expression. Reduced binding affinity resulting from mismatches may advantageously be compensated by increased number of nucleotides in the oligonucleotide and/or an increased number of modified nucleosides capable of increasing the binding affinity to the target, such as 2′ sugar modified nucleosides, including LNA, present within the oligonucleotide sequence.

Advantageously, the antisense oligonucleotide is capable of reducing the expression of ApoE4 more than it reduces the expression of ApoE3. In some embodiments, the antisense oligonucleotide is capable of reducing the expression of ApoE4 such that, in a target cell comprising both ApoE4 nucleic acid and ApoE3 nucleic acid, the ratio of the percentage of remaining ApoE3 nucleic acid (as compared to a control) and the percentage of remaining ApoE4 nucleic acid (as compared to a control) is higher than 1, preferably at least 1.5, more preferably at least 2, at least 2.5, at least 3, at least 3.5, or at least 4. the normal expression level of the ApoE3 nucleic acid).

In some embodiments, the antisense oligonucleotide is capable of reducing the expression level of ApoE4 mRNA such that, in a target cell comprising both ApoE4 mRNA and ApoE3 mRNA, the ratio of the percentage of remaining ApoE3 mRNA (as compared to a control) and the percentage of remaining ApoE4 mRNA (as compared to a control) is higher than 1, preferably at least 1.5, more preferably at least 2, at least 2.5, at least 3, at least 3.5, or at least 4 following application of 25 μM oligonucleotide to neuroblastoma cells having a heterozygous genotype of APOE ε3 and APOE ε4.

In particular, the antisense oligonucleotide may be capable of reducing the expression of ApoE4 such that, in a target cell comprising
(a) mRNA which encodes a human ApoE4 protein encoded by SEQ ID NO:1 and comprises a segment corresponding to positions 516 to 556 of SEQ ID NO:1, and
(b) mRNA which encodes a human ApoE3 protein encoded by SEQ ID NO:2 and comprises a segment corresponding to positions 516 to 556 of SEQ ID NO:2,
   the antisense oligonucleotide reduces the level of mRNA encoding human ApoE4 such that the ratio between
(i) the percentage of the level of remaining ApoE3 mRNA as compared to a control, and
(ii) the percentage of the level of remaining ApoE4 mRNA as compared to a control,
   is higher than 1, such as at least 1.5, such as at least 2, such as at least 2.5, such as at least 3, such as at least 3.5, such as at least 4.

Suitable controls reflecting the normal expression levels of ApoE4 and/or ApoE3 nucleic acids include the expression levels of ApoE3 mRNA and ApoE4 mRNA in the absence of the antisense oligonucleotide, such as a target cell contacted only with vehicle (e.g., PBS or culture medium) or with a control oligonucleotide which is known not to target the target sequence as defined in the present specification, and preferably known not to have any off-target activity (i.e. not having any relevant effect on the expression of the cell/organism genome). Reference (control) values may also be known from literature. Suitable target cells include human KELLY neuroblastoma cells of heterozygous APOE ε3/ε4 genotype (Schaffer et al., Genes Nutr. 2014 January; 9(1)). Example 1 provides an assay which may be used to measure the relative inhibition of ApoE3 and ApoE4 mRNA inhibition, using human KELLY neuroblastoma cells.

An aspect of the present invention relates to an antisense oligonucleotide which comprises a contiguous nucleotide sequence of at least 10 nucleotides in length with at least 80% complementarity to SEQ ID NO:1. The antisense oligonucleotide may also or alternatively have at least 80% complementarity to SEQ ID NO:3. In some embodiments, the antisense oligonucleotide comprises a contiguous nucleotide sequence of at least 10 nucleotides in length with at least 80%, such as at least 81%, such as at least 82%, such as at least 83%, such as at least 84%, such as at least 85%, such as at least 86%, such as at least 87%, such as at least 88%, such as at least 89%, such as least 90%, such as at least 91%, such as at least 92%, such as at least 93%, such as at least 94%, such as at least 95%, such as at least 96%, such as at least 97%, such as at least 98%, such as at least 99%, such as 100% complementarity to SEQ ID NO:1. In some embodiments, the antisense oligonucleotide also or alternatively comprises a contiguous nucleotide sequence of at least 10 nucleotides in length with at least 80%, such as at least 81%, such as at least 82%, such as at least 83%, such as at least 84%, such as at least 85%, such as at least 86%, such as at least 87%, such as at least 88%, such as at least 89%, such as least 90%, such as at least 91%, such as at least 92%, such as at least 93%, such as at least 94%, such as at least 95%, such as at least 96%, such as at least 97%, such as at least 98%, such as at least 99%, such as 100% complementarity to SEQ ID NO:3.

In some embodiments, the oligonucleotide comprises a contiguous sequence of 10 to 30 nucleotides in length, which is at least 80%, such as at least 81%, such as at least 82%, such as at least 83%, such as at least 84%, such as at least 85%, such as at least 86%, such as at least 87%, such as at least 88%, such as at least 89%, such as least 90%, such as at least 91%, such as at least 92%, such as at least 93%, such as at least 94%, such as at least 95%, such as at least 96%, such as at least 97%, such as at least 98%, such as at least 99%, such as 100% complementary with a region of the target nucleic acid or with a target sequence.

It is advantageous if the oligonucleotide of the invention, or contiguous nucleotide sequence thereof is fully complementary (100% complementary) to a region of the target nucleic acid or with a target sequence.

In some embodiments, the oligonucleotide, or contiguous nucleotide sequence thereof, may comprise from zero to three mismatches compared to the target sequence it is complementary to, optionally selected from one mismatch, two mismatches, and three mismatches. For example, the oligonucleotide, or contiguous nucleotide sequence thereof, may comprise one or two mismatches between the oligonucleotide, or contiguous nucleotide sequence thereof, and the target sequence in the target nucleic acid. The mismatch is not in the nucleotide at position 535 of SEQ ID NO:1. Accordingly, the nucleotide of the contiguous nucleotide sequence which is complementary to the nucleotide at position 535 of SEQ ID NO:1 comprises a guanine (g) nucleobase.

In some embodiments the oligonucleotide comprises a contiguous nucleotide sequence of 8 to 50 nucleotides in length which is at least 80% complementary, such as at least 90% complementary, such as fully (or 100%) complementary, to a target sequence within positions 516 to 556 of SEQ ID NO:1, such as within positions 522-548 of SEQ ID NO: 1.

In some embodiments the oligonucleotide comprises a contiguous nucleotide sequence of 10 to 30 nucleotides in length which is at least 80% complementary, such as at least 90% complementary, such as fully (or 100%) complementary, to a target sequence within positions 516 to 556 of SEQ ID NO:1, such as within positions 522-548 of SEQ ID NO: 1.

In some embodiments the oligonucleotide sequence is 100% complementary to a corresponding target sequence present in SEQ ID NO: 1.

It is also advantageous if the antisense oligonucleotide is complementary to a target sequence selected from one of the regions listed in Table 2. In some embodiments the contiguous nucleotide sequence of the antisense oligonucleotide is at least 80% complementary to, or at least 90% complementary to, such as fully complementary to, a target sequence selected from R_4 to R_96. In some embodiments the oligonucleotide sequence is 100% complementary to a target sequence selected from R_25, R_40, R_46, R_66 and R_91 (Table 2).

The oligonucleotide may comprise or consist of 8, 9, 10, 11, 12, 13, 14, 15, 16, 17, 18, 19, 20, 21, 22, 23, 24, 25, 26, 27, 28, 29, 30, 31, 32, 33, 3, 35, 36, 37, 38, 39, 40, 41, 42, 43, 44, 45, 46, 47, 48 or 50 nucleotides in length. The oligonucleotide may, for example, consist of 8, 9, 10, 11, 12, 13, 14, 15, 16, 17, 18, 19, 20, 21, 22, 23, 24, 25, 26, 27, 28, 29, 30, 31, 32, 33, 3, 35, 36, 37, 38, 39, 40, 41, 42, 43, 44, 45, 46, 47, 48 or 50 nucleotides in length. In some embodiments, the oligonucleotide comprises or consists of 8 to 50, such as of 8 to 40, such as of 10 to 40, such as of 10 to 35 nucleotides in length, such as from 10 to 30, such as 11 to 25, such as from 12 to 22, such as from 14 to 20 or 14 to 18 contiguous nucleotides in length. In one embodiment, the oligonucleotide comprises or consists of 16 to 22 nucleotides in length. In a preferred embodiment, the oligonucleotide comprises or consists of 16 to 20 nucleotides in length.

In some embodiments, the oligonucleotide or contiguous nucleotide sequence thereof comprises or consists of 22 or less nucleotides, such as 20 or less nucleotides, such as 16, 17, 18, 19 or 20 nucleotides. It is to be understood that any range given herein includes the range endpoints. Accordingly, if an oligonucleotide is said to include from 10 to 30 nucleotides, both 10 and 30 nucleotides are included.

In some embodiments, the contiguous nucleotide sequence comprises or consists of 10, 11, 12, 13, 14, 15, 16, 17, 18, 19, 20, 21, 22, 23, 24, 25, 26, 27, 28, 29 or 30 contiguous nucleotides in length. In a preferred embodiment, the oligonucleotide comprises or consists of 16, 17, 18, 19 or 20 nucleotides in length.

Preferably, the contiguous nucleotide sequence is 100% complementary to the target nucleic acid.

TABLE 3
Examples of motif sequences for
oligonucleotide or contiguous
nucleotide sequences

| | | Position | |
|---|---|---|---|
| | | of target | |
| | | sequence | |
| SEQ | | in SEQ | |
| ID | Motif sequence | ID NO: 1 | |
| NO | (5′-3′) | Start | Stop |
| 4 | GCACGTCCTCCATGTCCGCG | 516 | 535 |
| 5 | CGCACGTCCTCCATGTCCGC | 517 | 536 |
| 6 | GCACGTCCTCCATGTCCGC | 517 | 535 |
| 7 | CGCGCACGTCCTCCATGTCC | 518 | 538 |
| 8 | GCGCACGTCCTCCATGTCCG | 518 | 537 |
| 9 | CGCACGTCCTCCATGTCCG | 518 | 536 |
| 10 | GCACGTCCTCCATGTCCG | 518 | 535 |
| 11 | CGCGCACGTCCTCCATGTC | 519 | 538 |
| 12 | GCGCACGTCCTCCATGTCC | 519 | 537 |
| 13 | CGCACGTCCTCCATGTCC | 519 | 536 |
| 14 | GCACGTCCTCCATGTCC | 519 | 535 |
| 15 | CCGCGCACGTCCTCCATGTC | 520 | 539 |
| 16 | CGCGCACGTCCTCCATGT | 520 | 538 |
| 17 | GCGCACGTCCTCCATGTC | 520 | 537 |
| 18 | CGCACGTCCTCCATGTC | 520 | 536 |
| 19 | GCACGTCCTCCATGTC | 520 | 535 |
| 20 | GCCGCGCACGTCCTCCATGT | 521 | 540 |
| 21 | CCGCGCACGTCCTCCATGT | 521 | 539 |
| 22 | CGCGCACGTCCTCCATG | 521 | 538 |
| 23 | GCGCACGTCCTCCATGT | 521 | 537 |
| 24 | CGCACGTCCTCCATGT | 521 | 536 |
| 25 | GCACGTCCTCCATG | 522 | 535 |
| 26 | GGCCGCGCACGTCCTCCATG | 522 | 541 |
| 27 | GCCGCGCACGTCCTCCATG | 522 | 540 |
| 28 | CCGCGCACGTCCTCCATG | 522 | 539 |
| 29 | GCGCACGTCCTCCATG | 522 | 537 |
| 30 | CGGCCGCGCACGTCCTCCAT | 523 | 542 |
| 31 | GGCCGCGCACGTCCTCCAT | 523 | 541 |
| 32 | GCCGCGCACGTCCTCCAT | 523 | 540 |
| 33 | CCGCGCACGTCCTCCAT | 523 | 539 |
| 34 | CGCGCACGTCCTCCAT | 523 | 538 |
| 35 | GCGGCCGCGCACGTCCTCCA | 524 | 543 |
| 36 | CGGCCGCGCACGTCCTC | 524 | 542 |
| 37 | GGCCGCGCACGTCCTCCA | 524 | 541 |
| 38 | GCCGCGCACGTCCTCCA | 524 | 540 |
| 39 | CCGCGCACGTCCTCCA | 524 | 539 |
| 40 | GCGCACGTCCTCC | 525 | 537 |
| 41 | GGCGGCCGCGCACGTCCTCC | 525 | 544 |
| 42 | GCGGCCGCGCACGTCCTCC | 525 | 543 |
| 43 | CGGCCGCGCACGTCCTCC | 525 | 542 |
| 44 | GGCCGCGCACGTCCTCC | 525 | 541 |
| 45 | GCCGCGCACGTCCTCC | 525 | 540 |
| 46 | CGCGCACGTCCTC | 526 | 538 |
| 47 | AGGCGGCCGCGCACGTCCTC | 526 | 545 |
| 48 | GGCGGCCGCGCACGTCCTC | 526 | 544 |
| 49 | GCGGCCGCGCACGTCCTC | 526 | 543 |
| 50 | GGCCGCGCACGTCCTC | 526 | 541 |
| 51 | CAGGCGGCCGCGCACGTCCT | 527 | 546 |
| 52 | AGGCGGCCGCGCACGTCCT | 527 | 545 |
| 53 | GGCGGCCGCGCACGTCCT | 527 | 544 |
| 54 | GCGGCCGCGCACGTCCT | 527 | 543 |
| 55 | CGGCCGCGCACGTCCT | 527 | 542 |
| 56 | CCAGGCGGCCGCGCACGTCC | 528 | 547 |
| 57 | CAGGCGGCCGCGCACGTCC | 528 | 546 |
| 58 | AGGCGGCCGCGCACGTCC | 528 | 545 |
| 59 | GGCGGCCGCGCACGTCC | 528 | 544 |
| 60 | GCGGCCGCGCACGTCC | 528 | 543 |
| 61 | ACCAGGCGGCCGCGCACGTC | 529 | 548 |
| 62 | CCAGGCGGCCGCGCACGTC | 529 | 547 |
| 63 | CAGGCGGCCGCGCACGTC | 529 | 546 |
| 64 | AGGCGGCCGCGCACGTC | 529 | 545 |
| 65 | GGCGGCCGCGCACGTC | 529 | 544 |
| 66 | CAGGCGGCCGCGCACGT | 530 | 546 |
| 67 | CACCAGGCGGCCGCGCACGT | 530 | 549 |
| 68 | ACCAGGCGGCCGCGCACGT | 530 | 548 |
| 69 | CCAGGCGGCCGCGCACGT | 530 | 547 |
| 70 | AGGCGGCCGCGCACGT | 530 | 545 |
| 71 | GCACCAGGCGGCCGCGCACG | 531 | 550 |
| 72 | CACCAGGCGGCCGCGCACG | 53- | 549 |
| 73 | ACCAGGCGGCCGCGCACG | 53- | 548 |
| 74 | CCAGGCGGCCGCGCACG | 53- | 547 |
| 75 | CAGGCGGCCGCGCACG | 53- | 546 |
| 76 | TGCACCAGGCGGCCGCGCAC | 532 | 551 |
| 77 | GCACCAGGCGGCCGCGCAC | 532 | 550 |
| 78 | CACCAGGCGGCCGCGCAC | 532 | 549 |
| 79 | ACCAGGCGGCCGCGCAC | 532 | 548 |
| 80 | CCAGGCGGCCGCGCAC | 532 | 547 |
| 81 | CTGCACCAGGCGGCCGCGCA | 533 | 552 |
| 82 | TGCACCAGGCGGCCGCGCA | 533 | 551 |
| 83 | GCACCAGGCGGCCGCGCA | 533 | 550 |
| 84 | CACCAGGCGGCCGCGCA | 533 | 549 |
| 85 | ACCAGGCGGCCGCGCA | 533 | 548 |
| 86 | ACTGCACCAGGCGGCCGCGC | 534 | 553 |
| 87 | CTGCACCAGGCGGCCGCGC | 534 | 552 |
| 88 | TGCACCAGGCGGCCGCGC | 534 | 551 |
| 89 | GCACCAGGCGGCCGCGC | 534 | 550 |
| 90 | CACCAGGCGGCCGCGC | 534 | 549 |
| 91 | ACCAGGCGGCCGCG | 535 | 548 |
| 92 | TACTGCACCAGGCGGCCGCG | 535 | 554 |
| 93 | ACTGCACCAGGCGGCCGCG | 535 | 553 |
| 94 | CTGCACCAGGCGGCCGCG | 535 | 552 |
| 95 | TGCACCAGGCGGCCGCG | 535 | 551 |
| 96 | GCACCAGGCGGCCGCG | 535 | 550 |

In some embodiments, the oligonucleotide or contiguous nucleotide sequence comprises or consists of a sequence selected from those listed in Table 3.

In some embodiments, the antisense oligonucleotide or contiguous nucleotide sequence comprises or consists of 10 to 30 nucleotides in length with at least 90% identity, preferably 100% identity, to a sequence selected from the group consisting of SEQ ID NO: 4 to 96 (see motif sequences listed in Table 3).

In some embodiments, the antisense oligonucleotide or contiguous nucleotide sequence comprises or consists of 10 to 30 nucleotides in length with at least 90% identity, preferably 100% identity, to a sequence selected from the group consisting of SEQ ID NOS: 25, 40, 46, 66 and 91.

In some embodiments, the antisense oligonucleotide or contiguous nucleotide sequence comprises or consists of 10 to 30 nucleotides in length with at least 90% identity, preferably 100% identity, to a sequence selected from the group consisting of SEQ ID NO: 25, 40 and 46.

In some embodiments, the antisense oligonucleotide or contiguous nucleotide sequence comprises or consists of 10 to 30 nucleotides in length with at least 90% identity, preferably 100% identity, to a sequence of SEQ ID NO: 25.

In some embodiments, the antisense oligonucleotide or contiguous nucleotide sequence comprises or consists of 10 to 30 nucleotides in length with at least 90% identity, preferably 100% identity, to a sequence of SEQ ID NO: 40.

In some embodiments, the antisense oligonucleotide or contiguous nucleotide sequence comprises or consists of 10 to 30 nucleotides in length with at least 90% identity, preferably 100% identity, to a sequence of SEQ ID NO: 46.

In some embodiments, the antisense oligonucleotide or contiguous nucleotide sequence comprises or consists of 10 to 30 nucleotides in length with at least 90% identity, preferably 100% identity, to a sequence of SEQ ID NO: 66.

In some embodiments, the antisense oligonucleotide or contiguous nucleotide sequence comprises or consists of 10 to 30 nucleotides in length with at least 90% identity, preferably 100% identity, to a sequence of SEQ ID NO: 91.

In some embodiments, the contiguous nucleotide sequence comprises a sequence selected from SEQ ID NO: 4 to 96.

In some embodiments, the contiguous nucleotide sequence consists of a sequence selected from SEQ ID NO: 4 to 96.

In some embodiments, the antisense oligonucleotide comprises a sequence selected from SEQ ID NO: 4 to 96.

In some embodiments, the antisense oligonucleotide consists of a sequence selected from SEQ ID NO: 4 to 96.

In some embodiments, the contiguous nucleotide sequence comprises or consists of a nucleotide sequence as set forth in SEQ ID NO: 25.

In some embodiments, the contiguous nucleotide sequence comprises or consists of of a nucleotide sequence as set forth in SEQ ID NO: 40.

In some embodiments, the contiguous nucleotide sequence comprises or consists of of a nucleotide sequence as set forth in SEQ ID NO: 46.

In some embodiments, the contiguous nucleotide sequence comprises or consists of a nucleotide sequence as set forth in SEQ ID NO: 66.

In some embodiments, the contiguous nucleotide sequence comprises or consists of a nucleotide sequence as set forth in SEQ ID NO: 91.

In some embodiments, the antisense oligonucleotide sequence comprises or consists of a nucleotide sequence as set forth in SEQ ID NO: 25.

In some embodiments, the antisense oligonucleotide sequence comprises or consists of of a nucleotide sequence as set forth in SEQ ID NO: 40.

In some embodiments, the antisense oligonucleotide sequence comprises or consists of of a nucleotide sequence as set forth in SEQ ID NO: 46.

In some embodiments, the antisense oligonucleotide sequence comprises or consists of a nucleotide sequence as set forth in SEQ ID NO: 66.

In some embodiments, the antisense oligonucleotide sequence comprises or consists of a nucleotide sequence as set forth in SEQ ID NO: 91.

It is understood that the contiguous nucleobase sequences (motif sequence) can be modified to for example increase nuclease resistance and/or binding affinity to the target nucleic acid.

The pattern in which the modified nucleosides (such as high affinity modified nucleosides) are incorporated into the oligonucleotide sequence is generally termed oligonucleotide design.

Advantageously, the oligonucleotide sequence does not contain RNA nucleosides since this will decrease nuclease resistance. Additionally, as described elsewhere herein, an antisense oligonucleotide may advantageously comprise one or more modified nucleosides or nucleotides, such as 2′ sugar modified nucleosides. Furthermore, it is advantageous that the nucleosides which are not modified are DNA nucleosides. Accordingly, the oligonucleotides of the invention can be designed with modified nucleosides and DNA nucleosides. Preferably, high affinity modified nucleosides are used.

In an embodiment, the oligonucleotide comprises at least 1 modified nucleoside, such as at least 2, at least 3, at least 4, at least 5, at least 6, at least 7, at least 8, at least 9, at least 10, at least 11, at least 12, at least 13, at least 14, at least 15 or at least 16 modified nucleosides. In an embodiment the oligonucleotide comprises from 1 to 10 modified nucleosides, such as from 2 to 9 modified nucleosides, such as from 3 to 8 modified nucleosides, such as from 4 to 7 modified nucleosides, such as 6 or 7 modified nucleosides. Suitable modifications are described in the “Definitions” section under “modified nucleoside”, “high affinity modified nucleosides”, “sugar modifications”, “2′ sugar modifications” and Locked nucleic acids (LNA)”.

In an embodiment, the oligonucleotide comprises one or more sugar modified nucleosides, such as 2′ sugar modified nucleosides. Preferably the oligonucleotide of the invention comprises one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA).

In a further embodiment the oligonucleotide comprises at least one modified internucleoside linkage. Suitable internucleoside modifications are described in the “Definitions” section under “Modified internucleoside linkage”. It is advantageous if at least 75%, such as 80%, such as all, the internucleoside linkages within the contiguous nucleotide sequence are phosphorothioate or boranophosphate internucleoside linkages. In some embodiments all the internucleotide linkages in the contiguous sequence of the oligonucleotide are phosphorothioate linkages.

In some embodiments, the oligonucleotide of the invention comprises at least one LNA nucleoside, such as 1, 2, 3, 4, 5, 6, 7, or 8 LNA nucleosides, such as from 2 to 6 LNA nucleosides, such as from 3 to 7 LNA nucleosides, 4 to 8 LNA nucleosides or 3, 4, 5, 6, 7 or 8 LNA nucleosides. In some embodiments, at least 75% of the modified nucleosides in the oligonucleotide are LNA nucleosides, such as 80%, such as 85%, such as 90% of the modified nucleosides are LNA nucleosides, in particular beta-D-oxy LNA or ScET. In a still further embodiment all the modified nucleosides in the oligonucleotide are LNA nucleosides. In a further embodiment, the oligonucleotide may comprise both beta-D-oxy-LNA, and one or more of the following LNA nucleosides: thio-LNA, amino-LNA, oxy-LNA, ScET and/or ENA in either the beta-D or alpha-L configurations or combinations thereof. In a further embodiment, all LNA cytosine units are 5-methyl-cytosine. It is advantageous for the nuclease stability of the oligonucleotide or contiguous nucleotide sequence to have at least 1 LNA nucleoside at the 5′ end and at least 2 LNA nucleosides at the 3′ end of the nucleotide sequence.

In an embodiment of the invention the oligonucleotide of the invention is capable of recruiting RNase H.

In the current invention an advantageous structural design is a gapmer design as described in the “Definitions” section under for example “Gapmer”, “LNA Gapmer”, “MOE gapmer” and “Mixed Wing Gapmer” “Alternating Flank Gapmer”. The gapmer design includes gapmers with uniform flanks, mixed wing flanks, alternating flanks, and gapbreaker designs.

It is advantageous if the oligonucleotide is a gapmer with an F-G-F′ design, particular gapmer of formula 5′-F-G-F′-3′, where region F and F′ independently comprise 1-8 nucleosides, of which 2-5 are 2′ sugar modified and defines the 5′ and 3′ end of the F and F′ region, and G is a region between 6 and 16 nucleosides which are capable of recruiting RNaseH, such as a region comprising 6-16 DNA nucleosides.

In some embodiments, both flanks have a 2′ sugar modified nucleoside at the 5′ and 3′ terminal.

In some embodiments the gapmer is an LNA gapmer.

In some embodiments of the invention the LNA gapmer is selected from the following uniform flank designs: 2-13-2, 2-8-3, 3-8-2, 2-9-3, 3-9-2, 2-8-4, and 2-9-2.

In some embodiments, the LNA gapmer has alternating flank designs. In alternating flank designs, at least one of the flanks (F or F′) comprises one or more DNA nucleosides in addition to the LNA nucleosides. The flanking region F or F′, or both F and F′, then comprise at least three nucleosides, and the 5′ and 3′ most nucleosides of the F and/or F′ region are LNA nucleosides. With each “-” (dash) representing a shift between LNA/DNA nucleosides, the number representing the number of nucleosides, and the highest number representing DNA nucleosides in the gap region G, a gapmer with an alternating flank design with F having 5 nucleosides which are LNA-LNA-DNA-DNA-LNA, the gap region having 6 DNA nucleosides, and F′ having 2 LNA nucleosides can thus be represented as 2-2-1-6-2.

Using the same representation, in some embodiments of the invention the LNA gapmer is selected from the following alternating flanks designs: 2-1-1-1-1-6-2, 2-7-1-1-2, 2-1-1-6-3, 2-1-2-6-2, 2-8- 1-1-2, 2-7-1-1-3, 2-7-1-2-2, 2-2-1-6-2 and 2-1-1-7-2.

Tables 4 and 5 lists preferred designs of each motif sequence.

In all instances the F-G-F′ design may further include region D′ and/or D″ as described in the “Definitions” section under “Region D′or D” in an oligonucleotide”. In some embodiments the oligonucleotide of the invention has 1, 2 or 3 phosphodiester linked nucleoside units, such as DNA units, at the 5′ or 3′ end of the gapmer region.

In some embodiments, the oligonucleotide of the invention comprises both phosphorothioate internucleoside linkages and at least one phosphodiester linkage, such as 2, 3 or 4 phosphodiester linkages, in addition to the phosphorodithioate linkage(s). In a gapmer oligonucleotide, phosphodiester linkages, when present, are suitably not located between contiguous DNA nucleosides in the gap region G.

Advantageously, all the internucleoside linkages of the contiguous nucleotide sequence of the oligonucleotide are phosphorothioate, or all the internucleoside linkages of the oligonucleotide are phosphorothioate linkages. Nuclease resistant linkages, such as phosphorothioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F′, where all the internucleoside linkages in region G may be phosphorothioate.

For some embodiments, the oligonucleotide is the oligonucleotide compound with CMP ID NO: 91_1. For certain embodiments, the oligonucleotide is the oligonucleotide compound with CMP ID NO: 66_1. For certain embodiments, the oligonucleotide is the oligonucleotide compound with CMP ID NO: 46_1. For certain embodiments, the oligonucleotide is the oligonucleotide compound with CMP ID NO: 46_2. For certain embodiments, the oligonucleotide is the oligonucleotide compound with CMP ID NO: 46_3. For certain embodiments, the oligonucleotide is the oligonucleotide compound with CMP ID NO: 46_4. For certain embodiments, the oligonucleotide is the oligonucleotide compound with CMP ID NO: 46_5. For certain embodiments, the oligonucleotide is the oligonucleotide compound with CMP ID NO: 25_1. For certain embodiments, the oligonucleotide is the oligonucleotide compound with CMP ID NO: 25_2. For certain embodiments, the oligonucleotide is the oligonucleotide compound with CMP ID NO: 25_3. For certain embodiments, the oligonucleotide is the oligonucleotide compound with CMP ID NO: 25_4. For certain embodiments, the oligonucleotide is the oligonucleotide compound with CMP ID NO: 25_5. For certain embodiments, the oligonucleotide is the oligonucleotide compound with CMP ID NO: 25_6. For certain embodiments, the oligonucleotide is the oligonucleotide compound with CMP ID NO: 40_1. For certain embodiments, the oligonucleotide is the oligonucleotide compound with CMP ID NO: 40_2. For certain embodiments, the oligonucleotide is the oligonucleotide compound with CMP ID NO: 40_3.

A particular advantageous antisense oligonucleotide in the context of the invention is an oligonucleotide compound selected from the group consisting of

(SEQ ID NO: 91; CMP ID NO: 91_1)
ACcAgGcggccgCG
(SEQ ID NO: 66; CMP ID NO: 66_1)
CAggcggccgcgcacGT
(SEQ ID NO: 46; CMP ID NO: 46_1)
CGcgcacgtCcTC
(SEQ ID NO: 46; CMP ID NO: 46_2)
CGcgcacgtcCTC
(SEQ ID NO: 46; CMP ID NO: 46_3)
CGcGcacgtcCTC
(SEQ ID NO: 46; CMP ID NO: 46_4)
CGCgcacgtccTC
(SEQ ID NO: 46; CMP ID NO: 46_5)
CGcGCacgtccTC
(SEQ ID NO: 25; CMP ID NO: 25_1)
GCacgtcctccATG
(SEQ ID NO: 25; CMP ID NO: 25_2)
GCAcgtcctccaTG
(SEQ ID NO: 25; CMP ID NO: 25_3)
GCacgtcctcCaTG
(SEQ ID NO: 25; CMP ID NO: 25_4)
GCacgtcctCcATG
(SEQ ID NO: 25; CMP ID NO: 25_5)
GCacgtcctCcaTG
(SEQ ID NO: 25; CMP ID NO: 25_6)
GCacgtcctcCATG
(SEQ ID NO: 40; CMP ID NO: 40_1)
GCgcacgtcctCC
(SEQ ID NO: 40; CMP ID NO: 40_2)
GCgcAcgtcctCC
(SEQ ID NO: 40; CMP ID NO: 40_3)
GCgCacgtcctCC

wherein capital letters are beta-D-oxy LNA nucleosides, lowercase letters are DNA nucleosides, all LNA C are 5-methyl cytosine, all internucleoside linkages are phosphorothioate internucleoside linkages.

A further particular advantageous oligonucleotide in the context of the invention is an oligonucleotide compound selected from the group consisting of the compounds defined in Table 4 by HELM annotations. That is, the structure of each compound is described by the hierarchical editing language for macromolecules (HELM) (for details, see Zhang et al., Chem. Inf. Model. 2012, 52, 10, 2796-2806 or J. Chem. Inf. Model. 2017, 57, 6, 1233-1239) using the following HELM annotation keys:
[LR](G) is a beta-D-oxy-LNA guanine nucleoside,
[LR](T) is a beta-D-oxy-LNA thymine nucleoside,
[LR](A) is a beta-D-oxy-LNA adenine nucleoside,
[LR]([5meC]) is a beta-D-oxy-LNA 5-methyl cytosine nucleoside,
[dR](G) is a DNA guanine nucleoside,
[dR](T) is a DNA thymine nucleoside,
[dR](A) is a DNA adenine nucleoside,
[dR]([C]) is a DNA cytosine nucleoside,
[sP] is a phosphorothioate internucleoside linkage (stereo-undefined)

Further information as well as open-source tools for HELM can be found at the internet addresses www.pistoiaalliance.org/helm-tools/and www.pistoiaalliance.org/membership/about/. In particular, in Table 4, the designations “RNA1” and “$$$$V2.0” represent information useful for computerized analysis of HELM sequences and are not to be regarded as limiting for the oligonucleotide sequence defined between the brackets (i.e., between “{” and “}”).

TABLE 4
Compounds defined by HELM annotations.

| CMP | |
|---|---|
| ID NO | HELM |
| 91_1 | RNA1{[LR](A)[sP].[LR]([5meC])[sP].[dR](C)[P].[LR](A)[sP].[dR](G)[sP].[LR](G)[sP].[dR](C)[sP]. |
| | [dR](G)[sP].[dR](G)[sP].[dR](C)[sP].[dR](C)[sP].[dR](G)[sP].[LR]([5meC])[sP].[LR](G)}$$$$V2.0 |
| 66_1 | RNA1{[LR]([5meC])[sP].[LR](A)[sP].[dR](G)[sP].[dR](G)[sP].[dR](C)[sP].[dR](G)[sP].[dR](G)[sP]. |
| | [dR](C)[sP].[dR](C)[sP].[dR](G)[sP].[dR](C)[sP].[dR](G)[sP].[dR](C)[sP].[dR](A)[sP].[dR](C)[sP]. |
| | [LR](G)[sP].[LR](T)}$$$$V2.0 |
| 46_1 | RNA1{[LR]([5meC])[sP].[LR](G)[sP].[dR](C)[sP].[dR](G)[sP].[dR](C)[sP].[dR](A)[sP].[dR](C)[sP]. |
| | [dR](G)[sP].[dR](T)[sP].[LR]([5meC])[sP].[dR](C)[sP].[LR](T)[sP].[LR]([5meC])}$$$$V2.0 |
| 46_2 | RNA1{[LR]([5meC])[sP].[LR](G)[sP].[dR](C)[sP].[dR](G)[sP].[dR](C)[sP].[dR](A)[sP].[dR](C)[sP]. |
| | [dR](G)[sP].[dR](T)[sP].[dR](C)[sP].[LR]([5meC])[sP].[LR](T)[sP].[LR]([5meC])}$$$$V2.0 |
| 46_3 | RNA1{[LR]([5meC])[sP].[LR](G)[sP].[dR](C)[sP].[LR](G)[sP].[dR](C)[sP].[dR](A)[sP].[dR](C)[sP]. |
| | [dR](G)[sP].[dR](T)[sP].[dR](C)[sP].[LR]([5meC])[sP].[LR](T)[sP].[LR]([5meC])}$$$$V2.0 |
| 46_4 | RNA1{[LR]([5meC])[sP].[LR](G)[sP].[LR]([5meC])[sP].[dR](G)[sP].[dR](C)[sP].[dR](A)[sP].[dR](C)[sP]. |
| | [dR](G)[sP].[dR](T)[sP].[dR](C)[sP].[dR](C)[sP].[LR](T)[sP].[LR]([5meC])}$$$$V2.0 |
| 46_5 | RNA1{[LR]([5meC])[sP].[LR](G)[sP].[dR](C)[sP].[LR](G)[sP].[LR]([5meC])[sP].[dR](A)[sP].[dR](C)[sP]. |
| | [dR](G)[sP].[dR](T)[sP].[dR](C)[sP].[dR](C)[sP].[LR](T)[sP].[LR]([5meC])}$$$$V2.0 |
| 25_1 | RNA1{[LR](G)[sP].[LR]([5meC])[sP].[dR](A)[sP].[dR](C)[sP].[dR](G)[sP].[dR](T)[sP].[dR](C)[sP]. |
| | [dR](C)[sP].[dR](T)[sP].[dR](C)[sP].[dR](C)[sP].[LR](A)[sP].[LR](T)[sP].[LR](G)}$$$$V2.0 |
| 25_2 | RNA1{[LR](G)[sP].[LR]([5meC])[sP].[LR](A)[sP].[dR](C)[sP].[dR](G)[sP].[dR](T)[sP].[dR](C)[sP]. |
| | [dR](C)[sP].[dR](T)[sP].[dR](C)[sP].[dR](C)[sP].[LR](A)[sP].[LR](T)[sP].[LR](G)}$$$$V2.0 |
| 25_3 | RNA1{[LR](G)[sP].[LR]([5meC])[sP].[dR](A)[sP].[dR](C)[sP].[dR](G)[sP].[dR](T)[sP].[dR](C)[sP]. |
| | [dR](C)[sP].[dR](T)[sP].[dR](C)[sP].[LR]([5meC])[sP].[dR](A)[sP].[LR](T)[P].[LR](G)}$$$$V2.0 |
| 25_4 | RNA1{[LR](G)[sP].[LR]([5meC])[sP].[dR](A)[sP].[dR](C)[sP].[dR](G)[sP].[dR](T)[P].[dR](C)[sP]. |
| | [dR](C)[sP].[dR](T)[sP].[LR]([5meC])[sP].[dR](C)[sP].[LR](A)[sP].[LR](T)[sP].[LR](G)}$$$$V2.0 |
| 25_5 | RNA1{[LR](G)[sP].[LR]([5meC])[sP].[dR](A)[sP].[dR](C)[sP].[dR](G)[sP].[dR](T)[sP].[dR](C)[sP]. |
| | [dR](C)[sP].[dR](T)[sP].[LR]([5meC])[sP].[dR](C)[sP].[dR](A)[sP].[LR](T)[sP].[LR](G)}$$$$V2.0 |
| 25_6 | RNA1{[LR](G)[sP].[LR]([5meC])[P].[dR](A)[sP].[dR](C)[sP].[dR](G)[P].[dR](T)[sP].[dR](C)[sP]. |
| | [dR](C)[sP].[dR](T)[sP].[LR]([5meC])[sP].[dR](C)[sP].[dR](A)[sP].[LR](T)[sP].[LR](G)}$$$$V2.0 |
| 40_1 | RNA1{[LR](G)[sP].[LR]([5meC])[sP].[dR](G)[sP].[dR](C)[sP].[dR](A)[sP].[dR](C)[sP].[dR](G)[sP]. |
| | [dR](T)[sP].[dR](C)[sP].[dR](C)[sP].[dR](T)[sP].[LR]([5meC])[sP].[LR]([5meC])}$$$$V2.0 |
| 40_2 | RNA1{[LR](G)[sP].[LR]([5meC])[sP].[dR](G)[P].[dR](C)[sP].[LR](A)[P].[dR](C)[sP].[dR](G)[sP]. |
| | [dR](T)[sP].[dR](C)[sP].[dR](C)[sP].[dR](T)[P].[LR]([5meC])[sP].[LR]([5meC])}$$$$V2.0 |
| 40_3 | RNA1{[LR](G)[sP].[LR]([5meC])[sP].[dR](G)[sP].[LR]([5meC])[sP].[dR](A)[sP].[dR](C)[sP].[dR](G)[sP]. |
| | [dR](T)[sP].[dR](C)[sP].[dR](C)[sP].[dR](T)[sP].[LR]([5meC])[sP].[LR]([5meC])}$$$$V2.0 |

### Method of Manufacture

In a further aspect, the invention provides methods for manufacturing the oligonucleotides of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment the method further comprises reacting the contiguous nucleotide sequence with a conjugating moiety (ligand) to covalently attach the conjugate moiety to the oligonucleotide. In a further aspect a method is provided for manufacturing the composition of the invention, comprising mixing the oligonucleotide or conjugated oligonucleotide of the invention with a pharmaceutically acceptable diluent, solvent, carrier, salt and/or adjuvant.

### Pharmaceutical Salt

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin, Organic Process Research & Development 2000, 4, 427-435 or in Ansel, In: Pharmaceutical Dosage Forms and Drug Delivery Systems, 6th ed. (1995), pp. 196 and 1456-1457. For example, the pharmaceutically acceptable salt of the compounds provided herein may be a sodium salt.

In a further aspect the invention provides a pharmaceutically acceptable salt of the antisense oligonucleotide or a conjugate thereof. In a preferred embodiment, the pharmaceutically acceptable salt is a sodium or a potassium salt.

### Pharmaceutical Composition

In a further aspect, the invention provides pharmaceutical compositions comprising any of the aforementioned oligonucleotides and/or oligonucleotide conjugates or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the oligonucleotide is used in the pharmaceutically acceptable diluent at a concentration of 50 -300 μM solution.

Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091.

Oligonucleotides or oligonucleotide conjugates of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

These compositions may be sterilized by conventional sterilization techniques or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some embodiments, the oligonucleotide or oligonucleotide conjugate of the invention is a prodrug. In particular, with respect to oligonucleotide conjugates the conjugate moiety is cleaved off the oligonucleotide once the prodrug is delivered to the site of action, e.g., the target cell.

### Applications

The oligonucleotides of the invention may be utilized as research reagents for, for example, diagnostics, therapeutics and prophylaxis.

In research, such oligonucleotides may be used to specifically modulate the synthesis of ApoE4 protein in cells (e.g., in vitro cell cultures) and experimental animals thereby facilitating functional analysis of the target or an appraisal of its usefulness as a target for therapeutic intervention. Typically, the target modulation is achieved by degrading or inhibiting the mRNA producing the protein, thereby prevent protein formation or by degrading or inhibiting a modulator of the gene or mRNA producing the protein.

If employing the oligonucleotide of the invention in research or diagnostics the target nucleic acid may be a cDNA or a synthetic nucleic acid derived from DNA or RNA.

The present invention provides an in vivo or in vitro method for modulating ApoE4 expression in a target cell which is expressing ApoE4, said method comprising administering an oligonucleotide of the invention in an effective amount to said cell.

In some embodiments, the target cell, is a mammalian cell in particular a human cell. The target cell may be an in vitro cell culture or an in vivo cell forming part of a tissue in a mammal. In preferred embodiments the target cell is present in the brain or central nervous system. In particular cells in the cerebal cortex, medulla/pons, midbrain, frontal cortex, brain stem, cerebellum, and spinal cord can be relevant target regions. For the treatment of AD, target reduction in the brain regions cerebal cortex, medulla/pons and midbrain are advantageous. For the treatment of PSP, such as PSP with AD-like pathology, target reduction in the brain regions medulla/pons and midbrain are advantageous. In particular, microglia, neurons, nerve cells, astrocytes, axons and basal ganglia are or contain relevant cell types.

In diagnostics the oligonucleotides may be used to detect and quantitate ApoE4 expression in cell and tissues by northern blotting, in-situ hybridisation or similar techniques.

For therapeutics, the oligonucleotides may be administered to an animal or a human, suspected of having a disease or disorder which can be treated by modulating the expression of ApoE4.

For therapeutics, the oligonucleotides may also be administered to an animal or a human at risk for developing a disease or disorder which can be treated by modulating the expression of ApoE4.

For therapeutics, the oligonucleotides may also be administered to an animal or a human diagnosed as having a disease or disorder which can be treated by modulating the expression of ApoE4.

In particular, the invention provides methods for treating or preventing a disease or disorder, comprising administering a therapeutically or prophylactically effective amount of an oligonucleotide, an oligonucleotide conjugate or a pharmaceutical composition of the invention to a subject suffering from, at risk for, or susceptible to the disease or disorder.

The invention also relates to an oligonucleotide, a composition or a conjugate as defined herein for use as a medicament.

The oligonucleotide, oligonucleotide conjugate or a pharmaceutical composition according to the invention is typically administered in an effective amount.

The invention also provides for the use of the oligonucleotide or oligonucleotide conjugate of the invention as described for the manufacture of a medicament for the treatment or prevention of a disease or disorder as referred to herein, or for a method of the treatment or prevention of a disorder as referred to herein.

The disease or disorder, as referred to herein, is associated with the expression of ApoE4. The therapeutic applications of the invention are preferably employed for treatment or prophylaxis against diseases or disorders caused by abnormal levels and/or activity of ApoE4.

Advantageously, the therapeutic applications of the invention are employed for treatment of subjects suffering from or at risk for such a disease or disorder which carry at least one copy of an APOE ε4 gene in the genome.

In some embodiments, the subject suffering from or at risk for the disease or disorder carries a copy of an APOE ε4 gene in the genome.

In some embodiments, the subject suffering from or at risk for the disease or disorder carries a copy of an APOE ε3 gene in the genome.

In some embodiments, the subject suffering from or at risk for the disease has the heterozygous genotype APO ε3/ε4.

In some embodiments, the subject suffering or at risk for from the disease has the heterozygous genotype APO ε2/ε4.

In some embodiments, the subject suffering from or at risk for the disease has the homozygous genotype APO ε4/ε4.

In some embodiments, the subject suffering from or at risk for the disease is homozygous for the APO ε4 gene.

In some embodiments, treatment is performed on a subject who is at risk for, suspected of having, or having been diagnosed with, a neurological disorder, such as a neurological disorder selected from the group consisting of neurodegenerative diseases including Alzheimer's disease (AD), fronto-temporal dementia (FTD), Pick's disease (PiD), progressive supranuclear palsy (PSP), movement disorders such as Parkinson's disease (PD), dementia with Lewy Bodies, dementia in Down's Syndrome, and Niemann-Pick Type C1 Disease.

In one embodiment, the invention relates to oligonucleotides, oligonucleotide conjugates or pharmaceutical compositions for use in the treatment of diseases or disorders selected from AD, FTD, PiD, PSP, movement disorders such as PD, dementia with Lewy Bodies, dementia in Down's Syndrome, and Niemann-Pick Type C1 Disease.

In certain embodiments the disease or disorder is AD. The AD may, for example, be late-onset AD (over 65 years of age). The AD may in some embodiments be AD without dominant AD mutations. The AD may be early-onset AD. The AD may also be AD, or AD-type pathology, in a progressive supranuclear palsy (PSP) patient.

In certain embodiments the disease or disorder is fronto-temporal dementia (FTD).

In certain embodiments, the disease or disorder is Pick's disease (PiD).

In certain embodiments, the disease or disorder is progressive supranuclear palsy (PSP).

In certain embodiments, the disease or disorder is a movement disorder. For example, the movement disorder may be Parkinson's disease (PD).

In certain embodiments, the disease or disorder is dementia with Lewy Bodies.

In certain embodiments, the disease or disorder is dementia in a subject with Down's Syndrome.

In certain embodiments, the disease or disorder is Niemann-Pick Type C1 Disease.

In certain embodiments, the disease or disorder is dementia. Optionally, the dementia is dementia in, or associated with, any one or more of AD, FTD, PiD, PSP, PD, dementia with Lewy Bodies, dementia in a subject with Down's Syndrome, or Niemann-Pick Type C1 Disease.

### Administration

The oligonucleotides or pharmaceutical compositions of the present invention may be administered via parenteral (such as, intravenous, subcutaneous, intra-muscular, intracerebral, intracerebroventricular intraocular, or intrathecal administration).

In some embodiments, the administration is via intrathecal administration.

Advantageously, e.g., for treatment of neurological disorders, the oligonucleotide or pharmaceutical compositions of the present invention are administered intrathecally or intracranially, e.g., via intracerebral or intraventricular administration.

The invention also provides for the use of the oligonucleotide or conjugate thereof, such as pharmaceutical salts or compositions of the invention, for the manufacture of a medicament wherein the medicament is in a dosage form for subcutaneous administration.

The invention also provides for the use of the oligonucleotide of the invention, or conjugate thereof, such as pharmaceutical salts or compositions of the invention, for the manufacture of a medicament wherein the medicament is in a dosage form for intrathecal administration.

The invention also provides for the use of the oligonucleotide or oligonucleotide conjugate of the invention as described for the manufacture of a medicament wherein the medicament is in a dosage form for intrathecal administration.

### Combination Therapies

In some embodiments the oligonucleotide, oligonucleotide conjugate or pharmaceutical composition of the invention is for use in a combination treatment with another therapeutic agent. The therapeutic agent can for example be the standard of care for the diseases or disorders described above.

```
Sequences
NM_001302690.2 with rs429358 (APOE4, mRNA)
SEQ ID NO: 1
      1 ggatggggag ataagagaag accaggaggg agttaaatag ggaatgggtt gggggcggct
    61 tggtaaatgt gctgggatta ggctgttgca gataatgcaa caaggcttgg aaggctaacc
  121 tgggactggc caatcacagg caggaagatg aaggttctgt gggctgcgtt gctggtcaca
  181 ttcctggcag gatgccaggc caaggtggag caagcggtgg agacagagcc ggagcccgag
  241 ctgcgccagc agaccgagtg gcagagcggc cagcgctggg aactggcact gggtcgcttt
  301 tgggattacc tgcgctgggt gcagacactg tctgagcagg tgcaggagga gctgctcagc
  361 tcccaggtca cccaggaact gagggcgctg atggacgaga ccatgaagga gttgaaggcc
  421 tacaaatcgg aactggagga acaactgacc ccggtggcgg aggagacgcg ggcacggctg
  481 tccaaggagc tgcaggcggc gcaggcccgg ctgggcgcgg acatggagga cgtgcgcggc
  541 cgcctggtgc agtaccgcgg cgaggtgcag gccatgctcg gccagagcac cgaggagctg
  601 cgggtgcgcc tcgcctccca cctgcgcaag ctgcgtaagc ggctcctccg cgatgccgat
  661 gacctgcaga agcgcctggc agtgtaccag gccggggccc gcgagggcgc cgagcgcggc
  721 ctcagcgcca tccgcgagcg cctggggccc ctggtggaac agggccgcgt gcgggccgcc
  781 actgtgggct ccctggccgg ccagccgcta caggagcggg cccaggcctg gggcgagcgg
  841 ctgcgcgcgc ggatggagga gatgggcagc cggacccgcg accgcctgga cgaggtgaag
  901 gagcaggtgg cggaggtgcg cgccaagctg gaggagcagg cccagcagat acgcctgcag
  961 gccgaggcct tccaggcccg cctcaagagc tggttcgagc ccctggtgga agacatgcag
1021 cgccagtggg ccgggctggt ggagaaggtg caggctgccg tgggcaccag cgccgcccct
1081 gtgcccagcg acaatcactg aacgccgaag cctgcagcca tgcgacccca cgccaccccg
1141 tgcctcctgc ctccgcgcag cctgcagcgg gagaccctgt ccccgcccca gccgtcctcc
1201 tggggtggac cctagtttaa taaagattca ccaagtttca cgca
NM_001302690.2 (APOE3, mRNA)
SEQ ID NO: 2
      1 ggatggggag ataagagaag accaggaggg agttaaatag ggaatgggtt gggggcggct
    61 tggtaaatgt gctgggatta ggctgttgca gataatgcaa caaggcttgg aaggctaacc
  121 tgggactggc caatcacagg caggaagatg aaggttctgt gggctgcgtt gctggtcaca
  181 ttcctggcag gatgccaggc caaggtggag caagcggtgg agacagagcc ggagcccgag
  241 ctgcgccagc agaccgagtg gcagagcggc cagcgctggg aactggcact gggtcgcttt
  301 tgggattacc tgcgctgggt gcagacactg tctgagcagg tgcaggagga gctgctcagc
  361 tcccaggtca cccaggaact gagggcgctg atggacgaga ccatgaagga gttgaaggcc
  421 tacaaatcgg aactggagga acaactgacc ccggtggcgg aggagacgcg ggcacggctg
  481 tccaaggagc tgcaggcggc gcaggcccgg ctgggcgcgg acatggagga cgtgtgcggc
  541 cgcctggtgc agtaccgcgg cgaggtgcag gccatgctcg gccagagcac cgaggagctg
  601 cgggtgcgcc tcgcctccca cctgcgcaag ctgcgtaagc ggctcctccg cgatgccgat
  661 gacctgcaga agcgcctggc agtgtaccag gccggggccc gcgagggcgc cgagcgcggc
  721 ctcagcgcca tccgcgagcg cctggggccc ctggtggaac agggccgcgt gcgggccgcc
  781 actgtgggct ccctggccgg ccagccgcta caggagcggg cccaggcctg gggcgagcgg
  841 ctgcgcgcgc ggatggagga gatgggcagc cggacccgcg accgcctgga cgaggtgaag
  901 gagcaggtgg cggaggtgcg cgccaagctg gaggagcagg cccagcagat acgcctgcag
  961 gccgaggcct tccaggcccg cctcaagagc tggttcgagc ccctggtgga agacatgcag
1021 cgccagtggg ccgggctggt ggagaaggtg caggctgccg tgggcaccag cgccgcccct
1081 gtgcccagcg acaatcactg aacgccgaag cctgcagcca tgcgacccca cgccaccccg
1141 tgcctcctgc ctccgcgcag cctgcagcgg gagaccctgt ccccgcccca gccgtcctcc
1201 tggggtggac cctagtttaa taaagattca ccaagtttca cgca
XM_005589554.2 (macaca fascicularis apolipoprotein E (APOE), mRNA)
SEQ ID NO: 3
      1 atgagctcag gcgcctctag aaagtgtagc tgggacctcg ggaagccctg gcctccagac
    61 tggccaatca caggcaggaa gatgaaggtt ctgtgggctg cgttgctggt cacattcctg
  121 gcaggatgcc aggccaaggt ggagcaaccg gtggagccag agacggaacc cgagcttcgc
  181 cagcaggctg aggggcagag cggccagccc tgggagctgg cactgggtcg cttttgggat
  241 tacctgcgct gggtgcagac actgtctgag caggtgcagg aggagctgct cagcccccag
  301 gtcacccagg aactgacgac gctgatggat gagaccatga aggagttgaa ggcctacaaa
  361 tcggaactgg aggaacagct gagcccggtg gcggaggaga cgcgggcacg gctgtccaag
  421 gagctgcagg cggcgcaggc ccggctgggt gccgacatgg aggacgtgcg cagccgcctg
  481 gtgcagtacc gcagcgaggt gcaggccatg ctgggccaga gtaccgagga gctgcgggcg
  541 cgcctcgcct cccacctgcg caagctgcgc aagcggctcc tccgcgatgc tgatgacctg
  601 cagaagcgcc tggcagtgta tcaggccggg gcccgcgagg gcgccgagcg cggggtcagc
  661 gccatccgcg agcgcctggg acccctggtg gagcagggcc gcgtgcgggc cgccactgtg
  721 ggctccctgg ccagccagcc gcttcaggag cgggcccagg ccttgggtga gcggcttcgc
  781 gcacggatgg aggagatggg cagccggacc cgcgaccgcc tggacgaggt gaaggagcag
  841 gtggcggagg tgcgcgccaa gctggaggaa caggcccagc agataagcct gcaggccgag
  901 gccttccagg cccgcctcaa gagctggttc gagcccctcg tggaagatat gcagcgccag
  961 tgggctgggc tggtggagaa ggtgcaggct gccgtgggcg ccagcaccgc ccctgtgccc
1021 agcgacaatc actgaacgcc caggcctaca gccatgcgac ccgactccac cccatgcctc
1081 ctctctccgc tcagcctgca gcgggagacc ctgtccccgc cccagccgtc ctccaggggt
1141 gggccctagt ttaataaaga ttcgccaagt ttcaccgca
```

### EMBODIMENTS OF THE INVENTION

1. An antisense oligonucleotide of 8 to 50 nucleotides in length, such as of 10 to 30 nucleotides in length, which comprises a contiguous nucleotide sequence of at least 10 nucleotides in length which is at least 80% complementary to a target sequence within positions 516 to 556 of the Apolipoprotein (Apo) E4-encoding nucleic acid set forth as SEQ ID NO: 1, wherein the target sequence comprises position 535 of SEQ ID NO:1.
2. The antisense oligonucleotide according to item 1, wherein the contiguous nucleotide sequence has zero to three mismatches compared to the target sequence, optionally selected from one mismatch, two mismatches, and three mismatches, provided that the nucleotide of the contiguous nucleotide sequence which is complementary to the nucleotide at position 535 of SEQ ID NO:1 comprises a guanine (g) nucleobase.
3. The antisense oligonucleotide according to any one of the preceding items, wherein the contiguous nucleotide sequence is at least 90% complementary to a target sequence within positions 516 to 556 of SEQ ID NO: 1.
4. The antisense oligonucleotide according to any one of the preceding items, wherein the contiguous nucleotide sequence is 100% complementary to a target sequence within positions 516 to 556 of SEQ ID NO: 1.
5. The antisense oligonucleotide according to any one of the preceding items, wherein the contiguous nucleotide sequence is complementary to a target sequence within positions 522 to 548 of SEQ ID NO: 1.
6. The antisense oligonucleotide according to any one of the preceding items, wherein the contiguous nucleotide sequence is complementary to a target sequence selected from R_4 to R_96 in Table 2.
7. The antisense oligonucleotide according to any one of the preceding items, wherein the contiguous nucleotide sequence is complementary to a target sequence selected from residues 522 to 535 (R_25), residues 525 to 537 (R_40), residues 526 to 538 (R_46), residues 530 to 546 (R_66), and residues 535 to 548 (R_91) of SEQ ID NO:1.
8. The antisense oligonucleotide according to any one of the preceding items, wherein the contiguous nucleotide sequence comprises or consists of a nucleotide sequence selected from the group consisting of SEQ ID NO:4 to 96.
9. The antisense oligonucleotide according to any one of the preceding items, wherein the contiguous nucleotide sequence comprises or consists of a nucleotide sequence selected from the group consisting of SEQ ID NOS: 25, 40, 46, 66 and 91.
10. The antisense oligonucleotide according to any one of the preceding items, wherein the contiguous nucleotide sequence comprises or consists of SEQ ID NO:25.
11. The antisense oligonucleotide according to any one of the preceding items, wherein the contiguous nucleotide sequence comprises or consists of SEQ ID NO:40.
12. The antisense oligonucleotide according to any one of the preceding items, wherein the contiguous nucleotide sequence comprises or consists of SEQ ID NO:46.
13. The antisense oligonucleotide according to any one of the preceding items, wherein the contiguous nucleotide sequence comprises or consists of SEQ ID NO:66.
14. The antisense oligonucleotide according to any one of the preceding items, wherein the contiguous nucleotide sequence comprises or consists of SEQ ID NO:91.
15. The antisense oligonucleotide according to any one of the preceding items, wherein the antisense oligonucleotide is capable of reducing the expression of mammalian, such as human, Apolipoprotein (Apo) E4.
16. The antisense oligonucleotide according to any one of the preceding items, wherein the antisense oligonucleotide is capable of reducing the expression level of mRNA encoding mammalian, such as human, Apolipoprotein (Apo) E4 in a target cell by at least 20%, such as at least 30%, such as at least 40%, such as at least 50%, such as at least 60%, such as at least 70%, such as at least 80%, such as at least 90%, as compared to the normal expression level of the target.
17. The antisense oligonucleotide according to any one of the preceding items, wherein the antisense oligonucleotide is capable of reducing the expression of ApoE4 such that, in a target cell comprising
   (a) mRNA which encodes a human ApoE4 protein encoded by SEQ ID NO:1 and comprises a segment corresponding to positions 516 to 556 of SEQ ID NO:1, and
   (b) mRNA which encodes a human ApoE3 protein encoded by SEQ ID NO:2 and comprises a segment corresponding to positions 516 to 556 of SEQ ID NO:2,
      the antisense oligonucleotide reduces the level of mRNA encoding human ApoE4 such that the ratio between
   (i) the percentage of the level of remaining ApoE3 mRNA as compared to a control, and
   (ii) the percentage of the level of remaining ApoE4 mRNA as compared to a control, is higher than 1, such as at least 1.5, such as at least 2, such as at least 2.5, such as at least 3, such as at least 3.5, such as at least 4, optionally wherein the control is the level of mRNA in a control target cell in the absence of the antisense oligonucleotide.
18. The antisense oligonucleotide according to any one of the preceding items, wherein the target sequence is located in RNA.
19. The antisense oligonucleotide according to item 18, wherein the RNA is mRNA.
20. The antisense oligonucleotide according to item 19, wherein the mRNA is mature mRNA.
21. The antisense oligonucleotide according to any one of the preceding items, which comprises or consists of 10, 11, 12, 13, 14, 15, 16, 17, 18, 19, 20, 21, 22, 23, 24, 25, 26, 27, 28, 29 or 30 nucleotides in length.
22. The antisense oligonucleotide according to any one of the preceding items, which comprises or consists of 14 to 30 nucleotides in length.
23. The antisense oligonucleotide according to any one of the preceding items, which comprises or consists of 16 to 24 nucleotides in length.
24. The antisense oligonucleotide according to any one of the preceding items, wherein the contiguous nucleotide sequence has a length of 14 to 22 nucleotides.
25. The antisense oligonucleotide according to any one of the preceding items, wherein the contiguous nucleotide sequence has a length of at least 16 nucleotides, such as 16, 17, 18, 19, 20, 21 or 22 nucleotides.
26. The antisense oligonucleotide according to any one of the preceding items, which is single-stranded.
27. The antisense oligonucleotide according to any one of the preceding items, wherein the contiguous nucleotide sequence comprises one or more modified nucleosides, such as 2′ sugar modified nucleosides or Unlocked Nucleic Acids (UNA) nucleosides.
28. The antisense oligonucleotide according to any one of the preceding items, wherein the contiguous nucleotide sequence comprises one or more 2′ sugar modified nucleosides.
29. The antisense oligonucleotide according to item 28, wherein the one or more 2′ sugar modified nucleoside is independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and locked nucleic acid (LNA) nucleosides.
30. The antisense oligonucleotide according to item 29, wherein the one or more 2′ sugar modified nucleosides comprises at least one LNA nucleoside.
31. The antisense oligonucleotide according to item 30, wherein the at least one LNA nucleoside is independently selected from the group consisting of oxy-LNA, amino-LNA, thio-LNA, cET, and ENA LNA nucleosides.
32. The antisense oligonucleotide according to item 31, wherein the modified LNA nucleoside is oxy-LNA with the 2′-4′ bridge —O—CH₂—.
33. The antisense oligonucleotide according to item 32, wherein the oxy-LNA is beta-D-oxy-LNA.
34. The antisense oligonucleotide according to any one of items 29 to 33, wherein the contiguous nucleotide sequence comprises 4 to 8 LNA nucleosides.
35. The antisense oligonucleotide according to any one of the preceding items, which comprises at least one modified internucleoside linkage.
36. The antisense oligonucleotide according to any one of the preceding items, which comprises a nuclease resistant modified internucleoside linkage.
37. The antisense oligonucleotide according to any one of the preceding items, wherein at least 50% of the internucleoside linkages within the contiguous nucleotide sequence are phosphorothioate internucleoside linkages.
38. The antisense oligonucleotide according to any one of the preceding items, wherein at least 80% of the internucleoside linkages within the contiguous nucleotide sequence are phosphorothioate internucleoside linkages.
39. The antisense oligonucleotide according to any one of the preceding items, wherein the antisense oligonucleotide is capable of recruiting RNase H.
40. The antisense oligonucleotide according to anyone of the preceding items, wherein the oligonucleotide, or the contiguous nucleotide sequence thereof, is a gapmer.
41. The antisense oligonucleotide according to any one of the preceding items, wherein the oligonucleotide, or the contiguous nucleotide sequence thereof, is a gapmer of formula 5′-F-G-F′-3′, wherein each of region F and F′ independently comprises or consists of 1-8 nucleosides, of which 2 to 5 are 2′ sugar-modified nucleosides, and region G is a region of between 6 and 16 nucleosides which is capable of recruiting RNaseH.
42. The antisense oligonucleotide according to item 41, wherein region G is a region comprising 6 to 16 DNA nucleosides
43. The antisense oligonucleotide according to any one of items 41 and 42, wherein 2′ sugar-modified nucleosides define the 5′ and 3′ end of the F and F′ region.
44. The antisense oligonucleotide according to any one of items 41 to 43, wherein the 2′ sugar-modified nucleosides are according to any one of items 29 to 33.
45. The antisense oligonucleotide according to any one of items 41 to 44, wherein
   (a) the F region is between 2 and 8 nucleotides in length and consists of 2-5 identical LNA nucleosides and 0-4 DNA nucleosides; and
   (b) the F′ region is between 2 and 6 nucleotides in length and consists of 2-4 identical LNA nucleosides and 0-2 DNA nucleosides; and
   (c) region G is between 6 and 14 DNA nucleotides.
46. The antisense oligonucleotide according to any one of items 1 to 45, wherein the antisense oligonucleotide is or comprises a compound selected from the group consisting of:

(SEQ ID NO: 91; CMP ID NO: 91_1)
ACcAgGcggccgCG
SEQ ID NO: 66; CMP ID NO: 66_1)
CAggcggccgcgcacGT
SEQ ID NO: 46; CMP ID NO: 46_1)
CGcgcacgtCcTC
SEQ ID NO: 46; CMP ID NO: 46_2)
CGcgcacgtcCTC
SEQ ID NO: 46; CMP ID NO: 46_3)
CGcGcacgtcCTC
SEQ ID NO: 46; CMP ID NO: 46_4)
CGCgcacgtccTC
SEQ ID NO: 46; CMP ID NO: 46_5)
CGcGCacgtccTC
SEQ ID NO: 25; CMP ID NO: 25_1)
GCacgtcctccATG
SEQ ID NO: 25; CMP ID NO: 25_2)
GCAcgtcctccaTG
SEQ ID NO: 25; CMP ID NO: 25_3)
GCacgtcctcCaTG
SEQ ID NO: 25; CMP ID NO: 25_4)
GCacgtcctCcATG
SEQ ID NO: 25; CMP ID NO: 25_5)
GCacgtcctCcaTG
SEQ ID NO: 25; CMP ID NO: 25_6)
GCacgtcctcCATG
SEQ ID NO: 40; CMP ID NO: 40_1)
GCgcacgtcctCC
SEQ ID NO: 40; CMP ID NO: 40_2)
GCgcAcgtcctCC
SEQ ID NO: 40; CMP ID NO: 40_3)
GCgCacgtcctCC

wherein capital letters designate beta-D-oxy LNA nucleosides, lower case letters designate DNA nucleosides, capital C designate 5-methyl cytosine beta-D-oxy LNA nucleosides, and all internucleoside linkages are phosphorothioate internucleoside linkages.
47. The antisense oligonucleotide according to item 46, which is ACcAgGcggccgCG (SEQ ID NO:91; CMP ID NO:91_1).
48. The antisense oligonucleotide according to item 46, which is CAggcggccgcgcacGT (SEQ ID NO:66; CMP ID NO:66_1).
49. The antisense oligonucleotide according to item 46, which is CGcgcacgtCcTC (SEQ ID NO:46; CMP ID NO:46_1).
50. The antisense oligonucleotide according to item 46, which is CGcgcacgtcCTC (SEQ ID NO:46; CMP ID NO:46_2).
51. The antisense oligonucleotide according to item 46, which is CGcGcacgtcCTC (SEQ ID NO:46; CMP ID NO:46_3).
52. The antisense oligonucleotide according to item 46, which is CGCgcacgtccTC (SEQ ID NO:46; CMP ID NO:46_4).
53. The antisense oligonucleotide according to item 46, which is CGcGCacgtccTC (SEQ ID NO:46; CMP ID NO:46_5).
54. The antisense oligonucleotide according to item 46, which is GCacgtcctccATG (SEQ ID NO:25; CMP ID NO:25_1).
55. The antisense oligonucleotide according to item 46, which is GCAcgtcctccaTG (SEQ ID NO:25; CMP ID NO:25_2).
56. The antisense oligonucleotide according to item 46, which is GCacgtcctcCaTG (SEQ ID NO:25; CMP ID NO:25_3).
57. The antisense oligonucleotide according to item 46, which is GCacgtcctCcATG (SEQ ID NO:25; CMP ID NO:25_4).
58. The antisense oligonucleotide according to item 46, which is GCacgtcctCcaTG (SEQ ID NO:25; CMP ID NO:25_5).
59. The antisense oligonucleotide according to item 46, which is GCacgtcctcCATG (SEQ ID NO:25; CMP ID NO:25_6).
60. The antisense oligonucleotide according to item 46, which is GCgcacgtcctCC (SEQ ID NO:40; CMP ID NO:40_1).
61. The antisense oligonucleotide according to item 46, which is GCgcAcgtcctCC (SEQ ID NO:40; CMP ID NO:40_2),
62. The antisense oligonucleotide according to item 46, which is GCgCacgtcctCC (SEQ ID NO:40; CMP ID NO:40_3).
63. The antisense oligonucleotide according to any one of items 1 to 46, wherein one of the five, such as one of the four, such as one of the 1^{st}, 3^{rd} and 4^{th}, most 5′ nucleotides of the antisense oligonucleotide, or contiguous nucleotide sequence thereof, is complementary to position 535 in SEQ ID NO:1 and comprises a guanine nucleobase.
64. The antisense oligonucleotide according to any one of items 1 to 46, wherein one of the six, such as one of the 1^{st} and the 6^{th} most 3′ nucleotide of the antisense oligonucleotide, or contiguous nucleotide sequence thereof, is complementary to position 535 in SEQ ID NO:1 and comprises a guanine nucleobase.
65. The antisense oligonucleotide according to any one of items 1 to 46, wherein one of nucleotides of region G is complementary to position 535 in SEQ ID NO:1 and comprises a guanine nucleobase.
66. The antisense oligonucleotide according to any one of items 1 to 46, wherein one one of nucleotides of region F is complementary to position 535 in SEQ ID NO:1 and comprises a guanine nucleobase.
67. The antisense oligonucleotide according to any one of items 1 to 46, wherein one one of nucleotides of region F′ is complementary to position 535 in SEQ ID NO:1 and comprises a guanine nucleobase.
68. A conjugate comprising the antisense oligonucleotide according to any one of the preceding items and at least one conjugate moiety covalently attached to said oligonucleotide.
69. The conjugate according to item 68, wherein the conjugate moiety is selected from carbohydrates, cell surface receptor ligands, drug substances, hormones, lipophilic substances, polymers, proteins, peptides, toxins, vitamins, viral proteins or combinations thereof.
70. The conjugate according to item 69, wherein the conjugate moiety facilitates delivery across the blood brain barrier.
71. The conjugate according to item 70, wherein the conjugate moiety is an antibody or antibody fragment targeting the transferrin receptor.
72. The conjugate according to any one of items 68 to 70, comprising a linker which is positioned between the antisense oligonucleotide and the conjugate moiety.
73. The conjugate according to item 72, wherein the linker is a physiologically labile linker.
74. A pharmaceutically acceptable salt of the antisense oligonucleotide according to any one of items 1 to 67, or the conjugate according to any one of items 68 to 73.
75. A pharmaceutical composition comprising the antisense oligonucleotide according to any one of items 1 to 67, and/or the conjugate according to any one of items 68 to 73, and/or the pharmaceutically acceptable salt according to item 74; and a pharmaceutically acceptable diluent, solvent, carrier, salt and/or adjuvant.
76. A method for manufacturing the antisense oligonucleotide according to any one of items 1 to 67, comprising reacting nucleotide units thereby forming covalently linked contiguous nucleotide units comprised in the oligonucleotide.
77. The method according to item 76, further comprising reacting the contiguous nucleotide sequence with a non-nucleotide conjugation moiety.
78. A method for manufacturing the pharmaceutical composition according to item 75, comprising mixing the antisense oligonucleotide with a pharmaceutically acceptable diluent, carrier, salt and/or adjuvant.
79. A method for modulating ApoE4 expression in a target cell which is expressing ApoE4, said method comprising administering an antisense oligonucleotide according to any one of items 1 to 67, a conjugate according to any one of items 68 to 73 or a pharmaceutical composition according to item 74 in an effective amount to said cell.
80. The method according to item 79, which is an in vivo or in vitro method.
81. A method for treating or preventing a disease comprising administering a therapeutically or prophylactically effective amount of an antisense oligonucleotide according to any one of items 1 to 67, a conjugate according to any one of items 68 to 73, a pharmaceutically acceptable salt according to item 74, or a pharmaceutical composition according to item 75 to a subject suffering from or at risk for the disease.
82. An antisense oligonucleotide according to any one of items 1 to 67, a conjugate according to any one of items 68 to 73, a pharmaceutically acceptable salt according to item 74, or a pharmaceutical composition according to item 75, for use as a medicament.
83. An antisense oligonucleotide according to any one of items 1 to 67, a conjugate according to any one of items 68 to 73, a pharmaceutically acceptable salt according to item 74, or a pharmaceutical composition according to item 75, for use in a method of treating or preventing a disease.
84. Use of an antisense oligonucleotide according to any one of items 1 to 67, a conjugate according to any one of items 68 to 73, a pharmaceutically acceptable salt according to item 74, or a pharmaceutical composition according to item 75, for the preparation of a medicament for the treatment or prevention of a disease.
85. The method according to any one of items 79 to 81, the antisense oligonucleotide, conjugate, salt or pharmaceutical composition for use according to any one of items 82 and 83, or the use according to item 84, wherein the disease is associated with in vivo activity of ApoE4.
86. The method, the antisense oligonucleotide, conjugate, salt or pharmaceutical composition for use, or the use, according to item 85, wherein the in vivo activity of ApoE4 is reduced by at least 30%, such as at least 40%, such as at least 50%, such as at least 60%, such as at least 70%, such as at least 80%, such as at least 90%, compared to a control, optionally wherein the control is the in vivo activity of ApoE4 prior to administration of the antisense oligonucleotide, conjugate or pharmaceutical composition.
87. The method according to any one of items 79 to 81 and 85 to 86, the antisense oligonucleotide, conjugate, salt or pharmaceutical composition for use according to any one of items 82 and 83, or the use according to item 84, wherein the disease is associated with the expression level of ApoE4, optionally in a biological sample from the subject.
88. The method, the antisense oligonucleotide, conjugate, salt or pharmaceutical composition for use, or the use, according to item 87, wherein the expression level of ApoE4 is reduced by at least 30%, such as at least 40%, such as at least 50%, such as at least 60%, such as at least 70%, such as at least 80%, such as at least 90%, compared to a control, optionally wherein the control is the expression level of ApoE4 prior to administration of the antisense oligonucleotide, conjugate or pharmaceutical composition.
89. The method, the antisense oligonucleotide, conjugate, salt or pharmaceutical composition for use, or the use, according to any one of items 79 to 88, wherein the subject suffering from or at risk for the disease carries at least one copy of an APOE ε4 gene in the genome.
90. The method, the antisense oligonucleotide, conjugate, salt or pharmaceutical composition for use, or the use, according to item 89, wherein the subject suffering from or at risk for the disease is of the APOE ε3/ε4 genotype.
91. The method, the antisense oligonucleotide, conjugate, salt or pharmaceutical composition for use, or the use, according to item 89, wherein the subject suffering from or at risk for the disease is of the APOE ε4/ε4 genotype.
92. The method, the antisense oligonucleotide, conjugate, salt or pharmaceutical composition for use, or the use, according to any one of items 79 to 91, wherein the disease is a disease associated with dementia.
93. The method, the antisense oligonucleotide, conjugate, salt or pharmaceutical composition for use, or the use, according to any one of items 79 to 92, wherein the disease is selected from Alzheimer's disease (AD), fronto-temporal dementia (FTD), Pick's disease (PiD), progressive supranuclear palsy (PSP), movement disorders such as Parkinson's disease (PD), dementia with Lewy Bodies, dementia in Down's Syndrome, and Niemann-Pick Type C1 Disease.
94. The method, the antisense oligonucleotide, conjugate, salt or pharmaceutical composition for use, or the use, according to any one of items 79 to 93, wherein the disease is AD.
95. The method, the antisense oligonucleotide, conjugate, salt or pharmaceutical composition for use, or the use, according to any one of items 79 to 94, wherein the subject suffering from or at risk for the disease is a mammalian, such as a human, subject.

### EXAMPLES

### Materials and methods

### Oligonucleotide Motif Sequences and Oligonucleotide Compounds

TABLE 5
Compound Table
List of oligonucleotide motif sequences (indicated by SEQ ID NOs),
designs of these, as well as specific oligonucleotide compounds
(indicated by CMP ID NO) designed based on the motif sequence.

| | | | | | Start |
|---|---|---|---|---|---|
| | | | | | on |
| | | | | | SEQ |
| SEQ | | | | CMP | ID |
| ID | Motif sequence | | Oligonucleotide | ID | NO: |
| NO | (5′-3′) | Design | Compound | NO | 1 |
| 91 | accaggcggccgcg | 2-1-1-1-1-6-2 | ACcAgGcggccgCG | 91_1 | 535 |
| 66 | caggcggccgcgcacgt | 2-13-2 | CAggcggccgcgcacGT | 66_1 | 530 |
| 46 | cgcgcacgtcctc | 2-7-1-1-2 | CGcgcacgtCcTC | 46_1 | 526 |
| 46 | cgcgcacgtcctc | 2-8-3 | CGcgcacgtcCTC | 46_2 | 526 |
| 46 | cgcgcacgtcctc | 2-1-1-6-3 | CGcGcacgtcCTC | 46_3 | 526 |
| 46 | cgcgcacgtcctc | 3-8-2 | CGCgcacgtccTC | 46_4 | 526 |
| 46 | cgcgcacgtcctc | 2-1-2-6-2 | CGcGCacgtccTC | 46_5 | 526 |
| 25 | gcacgtcctccatg | 2-9-3 | GCacgtcctccATG | 25_1 | 522 |
| 25 | gcacgtcctccatg | 3-9-2 | GCAcgtcctccaTG | 25_2 | 522 |
| 25 | gcacgtcctccatg | 2-8-1-1-2 | GCacgtcctcCaTG | 25_3 | 522 |
| 25 | gcacgtcctccatg | 2-7-1-1-3 | GCacgtcctCcATG | 25_4 | 522 |
| 25 | gcacgtcctccatg | 2-7-1-2-2 | GCacgtcctCcaTG | 25_5 | 522 |
| 25 | gcacgtcctccatg | 2-8-4 | GCacgtcctcCATG | 25_6 | 522 |
| 40 | gcgcacgtcctcc | 2-9-2 | GCgcacgtcctCC | 40_1 | 525 |
| 40 | gcgcacgtcctcc | 2-2-1-6-2 | GCgcAcgtcctCC | 40_2 | 525 |
| 40 | gcgcacgtcctcc | 2-1-1-7-2 | GCgCacgtcctCC | 40_3 | 525 |

Motif sequences represent the contiguous sequence of nucleobases present in the oligonucleotide.

Designs refer to the gapmer design, F-G-F′, including alternating flanks as described elsewhere herein.

In the oligonucleotide compounds, a capital letter designates a beta-D-oxy LNA nucleoside, a lower-case letter designates a DNA nucleoside, a capital C designates a 5-methyl cytosine beta-D-oxy LNA nucleoside, and all internucleoside linkages are phosphorothioate internucleoside linkages. For a description of these compounds by HELM_annotations, see Table 4.

### Oligonucleotide Synthesis

Oligonucleotide synthesis is generally known in the art. Below is a protocol which may be applied. The oligonucleotide compounds described here may have been produced by slightly varying methods in terms of apparatus, support and concentrations used.

Oligonucleotides are synthesized on Unylinker universal solid supports (Org. Process Res. Dev. 2008, 12, 3, 399-410) using the phosphoramidite approach on an MermMade 192 oligonucleotide synthesizer at 1 μmol scale. At the end of the synthesis, the oligonucleotides are cleaved from the solid support using aqueous ammonia for 5-16 hours at 60° C. The oligonucleotides are purified by reverse phase HPLC (RP-HPLC) or by solid phase extractions and characterized by UPLC, and the molecular mass is further confirmed by ESI-MS.

### Elongation of the Oligonucleotide:

The coupling of 5′DMTr protected nucleoside β-cyanoethyl-phosphoramidites, including DNA-A(Bz), DNA-G(iBu), DNA-C(Bz), DNA-T, LNA-5-methyl-C(Bz), LNA-A(Bz), LNA-G(dmf), LNA-T, 2′OMe-A(Bz), 2′OMe(U), 2′OMe(T), 2′OMe-C(Ac), 2′OMe-G(iBu), 2′OMe-G(dmf), is performed by using a solution of 0.1 M of the 5′-O-DMT-protected phosphoramidite in acetonitrile and DCI (4,5-dicyanoimidazole) in acetonitrile (0.25 M) as activator.

### Purification by RP-HPLC:

The crude compounds are purified by preparative RP-HPLC on a Phenomenex Jupiter C18 10μ 150×10 mm column. 0.1 M ammonium acetate pH 8 and acetonitrile is used as buffers at a flow rate of 5 mL/min. The collected fractions are lyophilized to give the purified compound typically as a white solid.

### Abbreviations:

DCI: 4,5-Dicyanoimidazole
DCM: Dichloromethane
DMF: Dimethylformamidine
DMT: 4,4′-Dimethoxytrityl
THF: Tetrahydrofurane
Bz: Benzoyl
Ibu: Isobutyryl
RP-HPLC: Reverse phase high performance liquid chromatography

### Example 1: Fifteen Oligonucleotides Screened for Effects on APOE3 and APOE4 Expression Levels

Human KELLY neuroblastoma cells (ACC 355, DSMZ) were seeded in 96-well plates with 30000 cells per well in 190 ul of standard cell culture medium (RPMI-1640 Sigma R2405, 10% FBS, 25 μg/ml Penicillin-Streptomycin) the day prior to treatment. The KELLY cells were chosen based on their heterozygous genotype of APOE3 and APOE4 (Schaffer et al., Genes Nutr. 2014 Jan.; 9(1)). On the day of treatment oligonucleotides diluted in PBS (Gibco #14190-094) were added to obtain a gymnotic uptake with final concentrations in the media of 5 μM and 25 μM respectively in total volume of 200 μl per well.

Cells were incubated for 5 days at 37° C., 5% CO₂, and 98% humidity in an active evaporation incubator. At day 5 cell medium was removed from the culture wells by pipetting and RNA was extracted by adding 125 μL RLT buffer (Qiagen) and using RNeasy 96 kit and protocols from Qiagen. cDNA synthesis was performed using 4 μL input RNA was performed using IScript Advanced cDNA Synthesis Kit for RT-qPCR (Bio-Rad) and 2.5 μL was used as input for digital droplet PCR using ddPCR supermix for probes (no dUTP) (Bio-Rad) according to Manufacturer's protocol. The following assays were used:
APOE3/4: TaqMan@ SNP Genotyping Assay, rs429358 (Assay ID C_3084793_20, Thermo Fischer)
HPRT1: HPRT1 (ddPCR GEX CY5.5 assay 12005587 from Biorad)
APOE3 and APOE4 mRNA concentrations were quantified relative to the housekeeping gene HPRT1 using QuantaSoft Software (Bio-Rad).

Expression levels were then normalized to the average of untreated PBS control for APOE3 and APOE4, respectively. The results are shown in Table 6 where also the ratio of APOE3 versus APOE4 is included (high ratio indicate APOE4 selectivity).

TABLE 6
Data Table
Residual expression levels of APOE3 and APOE4 respectively
is shown as well as the ratio of APOE3 versus APOE4
(high ratio indicate APOE4 selectivity).

| | | | | | Ratio | Ratio |
|---|---|---|---|---|---|---|
| CMP ID | APOE3 | APOE3 | APOE4 | APOE4 | APOE3/APOE4 | APOE3/APOE4 |
| NO | (5 μM) | (25 μM) | (5 μM) | (25 μM) | (5 μM) | (25 μM) |
| 91_1 | ND | 12 | | 11 | N/A | 1.03 |
| 66_1 | 80 | 49 | 49 | 26 | 1.65 | 1.90 |
| 46_1 | 55 | 51 | 22 | 14 | 2.52 | 3.66 |
| 46_2 | 83 | 53 | 29 | 13 | 2.84 | 4.13 |
| 46_3 | 56 | 46 | 16 | 11 | 3.45 | 4.25 |
| 46_4 | 39 | 61 | 24 | 28 | 1.58 | 2.17 |
| 46_5 | 28 | 28 | 9 | 6 | 3.27 | 5.11 |
| 25_1 | 34 | 11 | 14 | 5 | 2.40 | 2.19 |
| 25_2 | 12 | 3 | 7 | 2 | 1.68 | 1.73 |
| 25_3 | 37 | 17 | 16 | 7 | 2.27 | 2.49 |
| 25_4 | 28 | 19 | 9 | 8 | 2.99 | 2.25 |
| 25_5 | 21 | 5 | 9 | 3 | 2.38 | 1.63 |
| 25_6 | 34 | 30 | 13 | 12 | 2.69 | 2.51 |
| 40_1 | 59 | 34 | 27 | 13 | 2.20 | 2.50 |
| 40_2 | 27 | 27 | 8 | 8 | 3.32 | 3.57 |
| 40_3 | 25 | 25 | 13 | 9 | 1.96 | 2.78 |

Various modifications and variations of the described aspects of the invention will be apparent to those skilled in the art without departing from the scope and spirit of the invention. Although the invention has been described in connection with specific preferred embodiments, the invention as claimed should not be unduly limited to such specific embodiments. Indeed, various modifications of the described modes of carrying out the invention which are obvious to those skilled in the relevant fields are intended to be within the scope of the following embodiments.

1. An antisense oligonucleotide of 8 to 50 nucleotides in length, such as of 10 to 30 nucleotides in length, which comprises a contiguous nucleotide sequence of at least 10 nucleotides in length which is at least 80% complementary to a target sequence within positions 516 to 556 of the Apolipoprotein (Apo) E4-encoding nucleic acid set forth as SEQ ID NO: 1, wherein the target sequence comprises position 535 of SEQ ID NO:1.

2. The antisense oligonucleotide according to claim 1, wherein the contiguous nucleotide sequence has zero to three mismatches compared to the target sequence, optionally selected from one mismatch, two mismatches, and three mismatches, provided that the nucleotide of the contiguous nucleotide sequence which is complementary to the nucleotide at position 535 of SEQ ID NO:1 comprises a guanine (g) nucleobase.

3. The antisense oligonucleotide according to any one of the preceding claims, wherein the contiguous nucleotide sequence is at least 90% complementary to a target sequence within positions 516 to 556 of SEQ ID NO: 1.

4. The antisense oligonucleotide according to any one of the preceding claims, wherein the contiguous nucleotide sequence is 100% complementary to a target sequence within positions 516 to 556 of SEQ ID NO: 1.

5. The antisense oligonucleotide according to any one of the preceding claims, wherein the contiguous nucleotide sequence is complementary to a target sequence within positions 522 to 548 of SEQ ID NO: 1.

6. The antisense oligonucleotide according to any one of the preceding claims, wherein the contiguous nucleotide sequence is complementary to a target sequence selected from residues 522 to 535 (R_25), residues 525 to 537 (R_40), residues 526 to 538 (R_46), residues 530 to 546 (R_66), and residues 535 to 548 (R_91) of SEQ ID NO:1.

7. The antisense oligonucleotide according to any one of the preceding claims, wherein the contiguous nucleotide sequence comprises or consists of a nucleotide sequence selected from the group consisting of SEQ ID NO: 4 to 96.

8. The antisense oligonucleotide according to any one of the preceding claims, wherein the contiguous nucleotide sequence comprises or consists of a nucleotide sequence selected from the group consisting of SEQ ID NOS: 25, 40, 46, 66 and 91.

9. The antisense oligonucleotide according to any one of the preceding claims, wherein the antisense oligonucleotide is capable of reducing the expression of mammalian, such as human, Apolipoprotein (Apo) E4.

10. The antisense oligonucleotide according to one of the preceding claims, wherein the antisense oligonucleotide is or comprises a compound selected from the group consisting of:
(SEQ ID NO: 91; CMP ID NO: 91_1)
ACcAgGcggccgCG
SEQ ID NO: 66; CMP ID NO: 66_1)
CAggcggccgcgcacGT
SEQ ID NO: 46; CMP ID NO: 46_1)
CGcgcacgtCcTC
SEQ ID NO: 46; CMP ID NO: 46_2)
CGcgcacgtcCTC
SEQ ID NO: 46; CMP ID NO: 46_3)
CGcGcacgtcCTC
SEQ ID NO: 46; CMP ID NO: 46_4)
CGCgcacgtccTC
SEQ ID NO: 46; CMP ID NO: 46_5)
CGcGCacgtccTC
SEQ ID NO: 25; CMP ID NO: 25_1)
GCacgtcctccATG
SEQ ID NO: 25; CMP ID NO: 25_2)
GCAcgtcctccaTG
SEQ ID NO: 25; CMP ID NO: 25_3)
GCacgtcctcCaTG
SEQ ID NO: 25; CMP ID NO: 25_4)
GCacgtcctCcATG
SEQ ID NO: 25; CMP ID NO: 25_5)
GCacgtcctCcaTG
SEQ ID NO: 25; CMP ID NO: 25_6)
GCacgtcctcCATG
SEQ ID NO: 40; CMP ID NO: 40_1)
GCgcacgtcctCC
SEQ ID NO: 40; CMP ID NO: 40_2)
GCgcAcgtcctCC
SEQ ID NO: 40; CMP ID NO: 40_3)
GCgCacgtcctCC
wherein capital letters designate beta-D-oxy LNA nucleosides, lower case letters designate DNA nucleosides, capital C designate 5-methyl cytosine beta-D-oxy LNA nucleosides, and all internucleoside linkages are phosphorothioate internucleoside linkages.

11. A conjugate comprising the antisense oligonucleotide according to any one of the preceding claims and at least one conjugate moiety covalently attached to said oligonucleotide.

12. A pharmaceutically acceptable salt of the antisense oligonucleotide according to any one of claims 1 to 10, or the conjugate according to claim 11.

13. A pharmaceutical composition comprising the antisense oligonucleotide according to any one of claims 1 to 10, and/or the conjugate according to claim 11, and/or the pharmaceutically acceptable salt according to claim 12; and a pharmaceutically acceptable diluent, solvent, carrier, salt and/or adjuvant.

14. An antisense oligonucleotide according to any one of claims 1 to 10, a conjugate according to claim 11, a pharmaceutically acceptable salt according to claim 12, or a pharmaceutical composition according to claim 13, for use in a method of treating or preventing a disease.

15. The antisense oligonucleotide, conjugate, salt or pharmaceutical composition for use according to claim 14, wherein the disease is associated with in vivo activity of ApoE4.

16. The antisense oligonucleotide, conjugate, salt or pharmaceutical composition for use according to claim 14 or 15, wherein the disease is a disease associated with dementia.

17. The antisense oligonucleotide, conjugate, salt or pharmaceutical composition for use according to any one of claims 14 to 16, wherein the disease is selected from Alzheimer's disease (AD), fronto-temporal dementia (FTD), Pick's disease (PiD), progressive supranuclear palsy (PSP), movement disorders such as Parkinson's disease (PD), dementia with Lewy Bodies, dementia in Down's Syndrome, and Niemann-Pick Type C1 Disease.

18. The antisense oligonucleotide, conjugate or pharmaceutical composition for use according to any one of claims 14 to 17, wherein the disease is AD.
